(19) United States
(12) Patent Application Publication
(10) Pub. No.: US 2024/0018082 A1
(43) Pub. Date: Jan. 18, 2024

(54) METAL FORMATE PRODUCTION

(71) Applicant: Twelve Benefit Corporation, Berkeley, CA (US)

(72) Inventors: Luka Stevic, Novi Sad (RS); Enrique Cintron, Brooklyn, NY (US); Lisa Wynia, Hayward, CA (US); Gregory DiCosola, Redmond, WA (US); Etosha R. Cave, Berkeley, CA (US); Myron Wayne Wilson, Jr., Longview, TX (US); Matthew Wayne Wilson, Longview, TX (US)

(21) Appl. No.: 18/328,540

(22) Filed: Jun. 2, 2023

Related U.S. Application Data
(60) Provisional application No. 63/367,100, filed on Jun. 27, 2022.

(51) Int. Cl. C07C 51/10; C25B 1/23

(52) U.S. Cl. CPC C07C 51/10; C25B 1/23

(57) ABSTRACT

Provided are systems and methods for metal formate synthesis from carbon monoxide supplied by an electrolyzer having a membrane electrode assembly which incorporate the capacity to recover and recycle various chemical components of the production process. For the synthesis, carbon monoxide from an electrolyzer is reacted with a solubilized metal hydroxide in a batch process or a continuous process.

### STATEMENT OF GOVERNMENT SUPPORT

This invention was made with government support under Grant Number FA864921P1530 awarded by the U.S. Air Force. The government has certain rights in the invention.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in their entireties and for all purposes.

### TECHNICAL FIELD

This disclosure relates generally to metal formate production, and more specifically to a new and useful system and method for advantageously utilizing carbon dioxide electrolyzer products for reaction with a metal hydroxide solubilized in methanol to form metal formates. The production process is configured for efficiently recycling components including intermediates, dryer gas, and methanol for re-use in metal formate production.

### BACKGROUND

Alkali metal formates have many uses including as enzyme stabilizers in liquid detergents. The enzymes may be lipases, amylases, proteases, etc. Other formates such as alkali earth metal formates also have many uses. Formate salts (MHCO₂, wherein M is typically Na, K or NH₄) and formic acid (HCO₂H) are commercial chemicals that can be produced by industrial thermochemical processes. For example, sodium formate and subsequently formic acid may be obtained by reaction of sodium hydroxide with carbon monoxide, followed by acidolysis with sulfuric acid.

Formic acid may also be produced as a co-product in the oxidation of hydrocarbons and by the hydrolysis of methyl formate from the carbonylation of methanol. Some techniques for the synthesis of formate salts by the electroreduction of carbon dioxide have also been described.

Since carbon dioxide is considered to be a cause of deleterious climate change, new and more robust methods to sequester CO₂ and/or efficiently convert it to useful products are still highly desirable in the quest to protect the environment while safeguarding valuable and limited resources.

The background description provided herein is for the purposes of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Provided are systems and methods for metal formate synthesis from carbon monoxide supplied by an electrolyzer having a membrane electrode assembly which incorporate the capacity to recover and recycle various chemical components of the production process. For the synthesis, carbon monoxide from an electrolyzer is reacted with a solubilized metal hydroxide in a batch process or a continuous process.

Accordingly, in a first aspect, the present invention encompasses a system for producing a metal formate including (a) a carbon dioxide reduction electrolyzer comprising a membrane electrode assembly, which comprises one or more ion conductive polymer layers and a cathode catalyst for facilitating chemical reduction of carbon dioxide to carbon monoxide; (b) at least one formate synthesis reactor configured to receive (i) a carbon monoxide feed produced by the carbon dioxide reduction electrolyzer, and (ii) a methanol feed comprising a metal hydroxide; (c) a metal formate separator configured to separate and purify metal formate produced by the formate synthesis reactor; and (d) a recycle path for recycling recovered methanol to the at least one formate synthesis reactor.

In some embodiments, the system also includes a carbon monoxide heater and a carbon monoxide compressor configured to heat and compress the carbon monoxide before introduction to the at least one formate synthesis reactor.

In some embodiments, the system also includes a methanol pump configured to transport methanol before introduction to the at least one formate synthesis reactor.

In some embodiments, the metal formate separator has a filter coupled to a dryer, wherein the filter is configured to receive an output from the at least one formate synthesis reactor, wherein the output includes the metal formate, methanol and optionally methyl formate, and wherein the dryer is configured with an inlet to receive the metal formate from the filter and an outlet for removing dried metal formate; and configured with a gas inlet for supplying a carrier gas and a gas outlet for removing dryer effluent gas containing methanol.

In some embodiments, the output is metal formate, methanol and methyl formate.

In some embodiments, the recycle path is a distillation column configured to separate liquid and/or gas output from the metal formate separator into recovered methanol and recovered methyl formate.

In some embodiments, the metal formate separator is a filter including a filtrate outlet connected to a distillation column, and wherein the distillation column is configured to produce recovered methyl formate and purified methanol.

In some embodiments, the system also includes a path for introducing the recovered methyl formate to the at least one formate synthesis reactor.

In some embodiments, the at least one formate synthesis reactor further includes an inlet for receiving the recovered methyl formate.

In some embodiments, the carrier gas is nitrogen gas.

In some embodiments, the system includes a condenser configured to purify the dryer effluent gas containing methanol to produce a purified carrier gas and a liquid methanol-containing process stream, wherein the purified carrier gas is recirculated to the dryer.

In some embodiments, the system includes a methanol separator coupled to the condenser, wherein the methanol separator is configured to separate and purify methanol from the liquid methanol-containing process stream to produce purified methanol.

In some embodiments, the methanol feed includes the purified methanol.

In some embodiments, the methanol separator is a distillation column, a moisture removal unit, or a combination thereof.

In some embodiments, the methanol separator is a moisture removal unit.

In some embodiments, the moisture removal unit is a packed bed of desiccant particles.

In some embodiments, the at least one formate synthesis reactor is a first formate synthesis reactor and a second formate synthesis reactor.

In some embodiments, the first formate synthesis reactor and the second formate synthesis reactor are batch reactors and are configured to be utilized alternatively.

In some embodiments, the at least one formate synthesis reactor is at least two formate synthesis reactors configured serially to operate continuously.

In some embodiments, the metal formate separator is a centrifuge.

In some embodiments, the at least one formate synthesis reactor is equipped with an agitator.

In some embodiments, the at least one formate synthesis reactor is equipped with a carbon monoxide inlet nozzle configured to increase reactivity of carbon monoxide.

In some embodiments, the metal hydroxide is sodium hydroxide, potassium hydroxide, rubidium hydroxide, cesium hydroxide, or a combination thereof.

In some embodiments, the system includes a mixing tank for solubilizing the metal hydroxide in methanol to produce the methanol feed, and the mixing tank is coupled to the at least one formate synthesis reactor through a pump.

In some embodiments, the carbon dioxide reduction electrolyzer and the at least one formate synthesis reactor are located in a single plant.

In some embodiments, the dryer and filter are combined into a single unit.

In some embodiments, the system also includes an oxalate synthesis reactor configured to convert the metal formate to a metal oxalate, and the system is further configured to transport the metal formate to the oxalate synthesis reactor.

In some embodiments, the system is further configured to contact the metal oxalate with an acid to thereby produce oxalic acid.

In some embodiments, the oxalate synthesis reactor is a pressure vessel.

In some embodiments, the oxalate synthesis reactor is configured to receive a metal carbonate catalyst for a reaction to convert the metal formate to the metal oxalate.

In a second aspect, the present invention encompasses a method of producing a metal formate including: (a) reducing carbon dioxide to carbon monoxide in a carbon dioxide reduction electrolyzer comprising a membrane electrode assembly, which comprises one or more ion conductive polymer layers and a cathode catalyst for facilitating chemical reduction of carbon dioxide to carbon monoxide; (b) reacting carbon monoxide produced by the carbon dioxide reduction electrolyzer with a metal hydroxide in methanol in at least one formate synthesis reactor to produce a metal formate; (c) separating and purifying the metal formate produced in (b); and (d) recycling methanol to the at least one formate synthesis reactor.

In some embodiments, the at least one formate synthesis reactor has at least two batch formate synthesis reactors and reacting carbon monoxide produced by the carbon dioxide reduction electrolyzer with a metal hydroxide in methanol is performed alternately in the at least two batch formate synthesis reactors.

In some embodiments, the at least one formate synthesis reactor includes at least two batch formate synthesis reactors and reacting carbon monoxide produced by the carbon dioxide reduction electrolyzer with a metal hydroxide in methanol is performed continuously in the at least two batch formate synthesis reactors.

In some embodiments, reacting the carbon monoxide with a metal hydroxide takes place at a reaction temperature of about 60° C. to about 200° C., and at a pressure of about 1 MPa to about 10 MPa.

In some embodiments, the metal hydroxide has a concentration of about 1 to about 25 percent by weight of metal hydroxide in the methanol.

In some embodiments, the method includes pre-heating the carbon monoxide from
(a) before reacting in (b).

In some embodiments, the carbon monoxide is pre-heated to at least about 100° C.

In some embodiments, the reaction temperature is about 100° C. to about 200° C.

In some embodiments, the method also includes producing methyl formate when reacting carbon monoxide with metal hydroxide in (b).

In some embodiments, the method also includes recovering the methyl formate by distillation and recycling the methyl formate to the at least one formate synthesis reactor.

In some embodiments, the method includes in (c) drying metal formate with a carrier gas, and producing dry metal formate and a dryer effluent gas stream.

In some embodiments, the method also includes condensing liquid from the dryer effluent gas stream to form a condensed liquid.

In some embodiments, the method also includes distilling the condensed liquid to obtain recovered methanol.

In some embodiments, recycling methanol to the at least one formate synthesis reactor includes recycling the recovered methanol to the formate synthesis reactor.

In some embodiments, (d) includes moisture removal.

In some embodiments, the method includes recovering an emitted carrier gas; and recycling the emitted carrier gas to the dryer.

In some embodiments, the carrier gas is nitrogen gas.

In some embodiments, the metal hydroxide is sodium hydroxide, potassium hydroxide, rubidium hydroxide, cesium hydroxide, or a combination thereof.

In some embodiments, the method also includes converting the metal formate to a metal oxalate.

In some embodiments, the method also includes contacting the metal oxalate with an acid to produce oxalic acid.

In some embodiments, the method also includes converting the metal formate to the metal oxalate is performed at a pressure above atmospheric pressure.

In some embodiments, converting the metal formate to the metal oxalate is by contacting the metal formate with a metal carbonate catalyst.

In a third aspect, the present invention encompasses a process for producing a metal formate, including (a) reducing carbon dioxide to carbon monoxide in a carbon dioxide reduction electrolyzer comprising a membrane electrode assembly, which comprises one or more ion conductive polymer layers and a cathode catalyst for facilitating chemical reduction of carbon dioxide to carbon monoxide; (b) mixing a metal hydroxide in methanol in a mixing tank to obtain solubilized metal hydroxide in methanol; (c) supplying carbon monoxide produced in (a) to at least one formate synthesis reactor, wherein the carbon monoxide is supplied to the formate synthesis reactor at a pressure of about 1 MPa to about 10 MPa, along with the solubilized metal hydroxide in methanol; (d) reacting the carbon monoxide with the solubilized metal hydroxide in methanol at a reaction temperature of about 60° C. to about 200° C., to produce a metal formate; (e) outputting from the at least one formate synthesis reactor a metal formate-containing stream comprising the metal formate, methanol and optionally methyl formate; (f) filtering the metal formate-containing stream produced in (e) to obtain solid metal formate, and filtrate comprising methanol and optionally methyl formate; (g) drying the solid metal formate produced in (f) in a dryer with a carrier gas to produce dried metal formate and an effluent gas stream comprising methanol and carrier gas; and (h) distilling the filtrate to produce methanol for recycling to the mixing tank and optionally methyl formate for feeding to the formate synthesis reactor.

In an embodiment, the process includes purifying the effluent gas stream of (g) to obtain recovered carrier gas and recycling the recovered carrier gas to the dryer.

In an embodiment, the solubilized metal hydroxide is about 1 to about 25 percent by weight of metal hydroxide in methanol.

In an embodiment, the process also includes, before (c), pre-heating the carbon monoxide produced in (a).

In some embodiments, the carbon monoxide produced in (a) is pre-heated to at least 100° C.

In some embodiments, the reaction temperature in (d) is about 100° C. to about 200° C.

In some embodiments, the carrier gas is nitrogen gas.

In some embodiments, the metal hydroxide is sodium hydroxide, potassium hydroxide, rubidium hydroxide, cesium hydroxide, or a combination thereof.

In a fourth aspect, the present disclosure encompasses a method of producing a metal formate. In some embodiments, the method includes (a) reducing carbon dioxide to carbon monoxide in a carbon dioxide reduction electrolyzer including a membrane electrode assembly, which has one or more ion conductive polymer layers and a cathode catalyst for facilitating chemical reduction of carbon dioxide to carbon monoxide; (b) reacting carbon monoxide produced by the carbon dioxide reduction electrolyzer with methanol in at least one methyl formate synthesis reactor to produce methyl formate and then separating and purifying the methyl formate; (c) hydrolyzing the methyl formate to formic acid with water in at least one methyl formate hydrolysis reactor and then separating and purifying the formic acid; (d) reacting the formic acid with a metal carbonate to produce a metal formate; and (e) recycling methanol from step (b) and water from step (c) to the at least one methyl formate synthesis reactor and recycling water from step (c) to the at least one methyl formate hydrolysis reactor; and wherein the metal formate is calcium formate or potassium formate; and the metal carbonate is calcium carbonate or potassium carbonate.

These and other features of the disclosure will be described in detail below with reference to associated figures.

### BRIEF DESCRIPTION OF THE FIGURES

FIG. 1 is a flow chart representation of an embodiment of the method, illustrating formate synthesis from carbon monoxide supplied from a CO₂ electrolyzer; with three component recycling.

FIG. 2 illustrates an examplary schematic formate production system comprising a carbon dioxide reduction electrolyzer, a formate production reactor, and various downstream formate recovery units.

FIG. 3 depicts an embodiment of the system and some associated components for producing metal formate using carbon monoxide from a carbon dioxide electrolyzer and parallel batch reactors.

FIG. 4 depicts an embodiment of the system and some associated components for producing metal formate using carbon monoxide from a carbon dioxide electrolyzer and parallel batch reactors.

FIG. 5 depicts an embodiment of the system and some associated components for continuous production of metal formate using carbon monoxide from a carbon dioxide electrolyzer, and serial continuous reactors.

### DETAILED DESCRIPTION

### Definitions

As used herein, the term “about” is understood to account for minor increases and/or decreases beyond a recited value, which changes do not significantly impact the desired function the parameter beyond the recited value(s). In some cases, “about” encompasses+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

As used herein, the phrase “at least one of A, B, and C” should be construed to mean a logical (A OR B OR C), using a non-exclusive logical OR, and should not be construed to mean ‘at least one of A, at least one of B, and at least one of C’.

As used herein, the phrase “recycle path” means any combination of condensation, purification and separation units necessary to prepare a reaction intermediate, byproduct, carrier gas or solvent for reuse, including means (e.g. delivery lines, compressors, pumps or the like) to return the recovered intermediate, byproduct, solvent and/or carrier gas to a reactor or other suitable re-entry point in the production process.

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

### 1. Overview

Aspects of this disclosure pertain to systems and methods that integrate a carbon dioxide reduction electrolyzer with one or more metal formate synthesis reactors. As explained elsewhere herein, the carbon dioxide reduction electrolyzer can be configured to efficiently reduce carbon dioxide to carbon monoxide. In certain embodiments, the carbon monoxide produced by such an electrolyzer is reacted with a metal hydroxide to produce a metal formate. This is performed in the one or more above-mentioned metal formate synthesis reactors.

In some implementations, the carbon monoxide from the carbon dioxide electrolyzer and metal hydroxide dissolved in methanol are provided to the one or more metal formate synthesis reactors. In these one or more reactors, the metal hydroxide and the carbon oxide react to form the metal formate.

In some embodiments, the system is designed or configured to preserve methanol by recovering it from a reaction stream, purifying it, and recycling it to dissolve fresh metal hydroxide before introduction to the one or more metal formate synthesis reactors.

In some embodiments, the system is designed or configured to dry solid metal formate produced by the one or more metal formate synthesis reactors using a drying gas. The system may be designed or configured to recycle the drying gas. In certain embodiments, the metal formate synthesis reaction, in the one or more metal formate synthesis reactors, produces methyl formate as an intermediate product. In such embodiments, the output stream of the one or more metal formate synthesis reactors includes some methyl formate. In such cases, the system may be designed or configured to separate recovered methanol and methyl formate before reusing the methanol in a recycle stream. In some implementations, methyl formate separated from methanol is fed directly back into the one or more metal formate synthesis reactors, while the purified methanol is first mixed with fresh metal hydroxide before reintroduction, along with dissolved metal hydroxide, to the one or more metal formate synthesis reactors.

Regarding the reaction of carbon monoxide with metal hydroxide in methanol, the solubility of carbon monoxide in methanol may limit the rate of the reaction. The reaction rate of carbon monoxide with metal hydroxide may exhibit second order reaction kinetics, dependent upon the concentration of carbon monoxide. Therefore, in some implementations, the integrated system and method include features to increase the concentration of carbon monoxide in methanol within the one or more metal formate synthesis reactors.

One approach to increasing the concentration of carbon monoxide involves increasing the pressure of carbon monoxide, above its pressure at the carbon dioxide electrolyzer, before introduction to the one or more metal formate synthesis reactors.

Another approach employs mixing the carbon monoxide gas with the methanol-containing reaction mixture in a manner that increases the interfacial surface area between carbon monoxide gas and methanol liquid. Various mechanisms may be employed for this purpose. In some embodiments, the one or more metal formate synthesis reactors operates with constant agitation to move carbon monoxide bubbles within the reaction mixture. Another approach employs a carbon monoxide introduction nozzle configured to introduce a carbon monoxide into the reaction mixture in a manner that maximizes the surface area of the interface between the carbon monoxide and the liquid phase. Reactor and nozzle designs that facilitate this high surface area contact are described elsewhere herein.

The formate synthesis reaction may be given by the following chemical equation:

CO+MOH→HCOOM

In certain embodiments, the reaction is conducted at an elevated temperature, such as about 100 to 200° C. Because the reaction is exothermic, the system may be configured in a manner that does not require preheating of methanol. In some embodiments, the metal formate synthesis reaction employs a relatively small amount of methanol. In some embodiments, a C₁-C₄ alcohol such as ethanol, propanol, isopropyl alcohol or butanol may be utilized as a solvent. In certain embodiments, the concentration of metal hydroxide in methanol is at least about 13 wt %. Note that the solubility limit of sodium hydroxide in methanol is about 23.1 wt %.

In some implementations, the carbon monoxide is preheated before introduction to the one or more metal formate synthesis reactors, while the methanol is not preheated before introduction to the reactors.

Given the relatively slow reaction rate of the metal formate synthesis reaction, the system may employ batch reactors in lieu of continuous reactors. Batch reactors present a throughput challenge in that non-reactor components of the system may need to sit idle while a batch reactor is refilled and the metal formate synthesis reaction is reinitiated. To address such a challenge, in some embodiments, the system employs two or more batch synthesis reactors. When employing two or more such reactors, the individual reactors can be operated out of phase, so that one is being recharged while the other is reacting. In this manner, metal formate can be produced continuously and fed continuously into downstream components of the system. In some embodiments, the system employs two or more continuous reactors arranged serially.

Methanol recycling is employed in certain implementations of the system. Some prior designs permitted methanol emission into the atmosphere during one or more process steps such as drying the metal formate reaction product. Commonly, air or nitrogen used in the drying process was emitted into the atmosphere. Methanol is a volatile organic compound which is permitted at a maximum concentration of 200 parts per million and flue gas per US OSHA regulations. While it is possible to combust volatile organic compounds such as methanol in flue gases, the required temperatures are typically about 400° C. Therefore, the combustion process is energy-intensive, and fossil fuels are regularly used to attain the necessary temperatures.

To reduce the environmental impact of the formate synthesis reaction, the system may be configured as a closed system, where nitrogen or other drying gas constantly re-circulates and methanol in the drying gas stream is condensed without being emitted or combusted. Methanol recovered in this manner may be recycled into the formate synthesis reactors.

In certain embodiments, nitrogen is employed as a drying gas because methanol is highly combustible. In some of limitations, the system employs a rotary dryer to evaporate the methanol from solid metal formate. The methanol that leaves in the nitrogen stream is condensed in a condenser and the newly dried nitrogen is recirculated back to the rotary dryers inlet.

In some reactors, methyl formate is an intermediate product in the reaction of carbon monoxide and metal hydroxide in methanol. In some embodiments, the liquid outlet stream of the metal formate synthesis reaction may contain about 5 to 10 wt % methyl formate.

In various embodiments, a liquid component of the reactor outlet stream is processed to separate methanol from methyl formate. In some implementations, the separation is accomplished by distillation. In some embodiments, the metal formate stream is directly fed back into the metal formate synthesis reactor where it may react with metal hydroxide to produce metal formate. In some implementations, the purified methanol, which has had most, or all, of its methyl formate removed, is fed back to a mixer where it solubilizes fresh metal hydroxide before that hydroxide is introduced into the one or more metal formate synthesis reactors.

A system and/or method for carbon dioxide reactor control may be configured to control aspects of reactor production, such as aspects relating to quantity, concentration, and/or ratios of reactor products. Electrochemical carbon oxide reduction cells may be integrated with any of various other chemical processing systems such as chemical reactors, chemical separation units, purification units, and the like, along with associated sensing and/or control systems. Integrated systems may employ an electrochemical carbon oxide reduction cell and another chemical processing system disposed upstream, downstream, or in parallel with the electrochemical carbon oxide reduction cell.

Examples of carbon oxide reactants include carbon dioxide and carbon monoxide, typically though not necessarily in gaseous form. Other examples of carbon oxide reactant include carbonate ions and compound, and bicarbonate ions and compounds.

Typical systems and methods for carbon dioxide reactor control have focused on maximization of aspects relating to production of carbon monoxide (CO) and/or other carbon-containing products (CCPs) (e.g., carbon-containing species (CCSs)), such as maximizing ratios of CO to other reactor products (e.g., CO:H₂ ratio), CO concentration, and/or total CO output or output rate.

However, simply maximizing aspect values can be undesirable, and that arbitrary control of such aspects (e.g., dynamic or selective aspect control to meet a value within a range of target aspect values), rather than simple maximization, can be beneficial. For example, it can be desirable to selectively control the CO:H₂ ratio of the reactor products (e.g., enabling arbitrary control within a spectrum from the highest CO:H₂ ratio possible for a given system and/or process, down to approximately 1:3 CO:H₂ or lower). With such control, the reactor output can be more effectively used (e.g., wherein the reactor outputs are directly fed to a subsequent input).

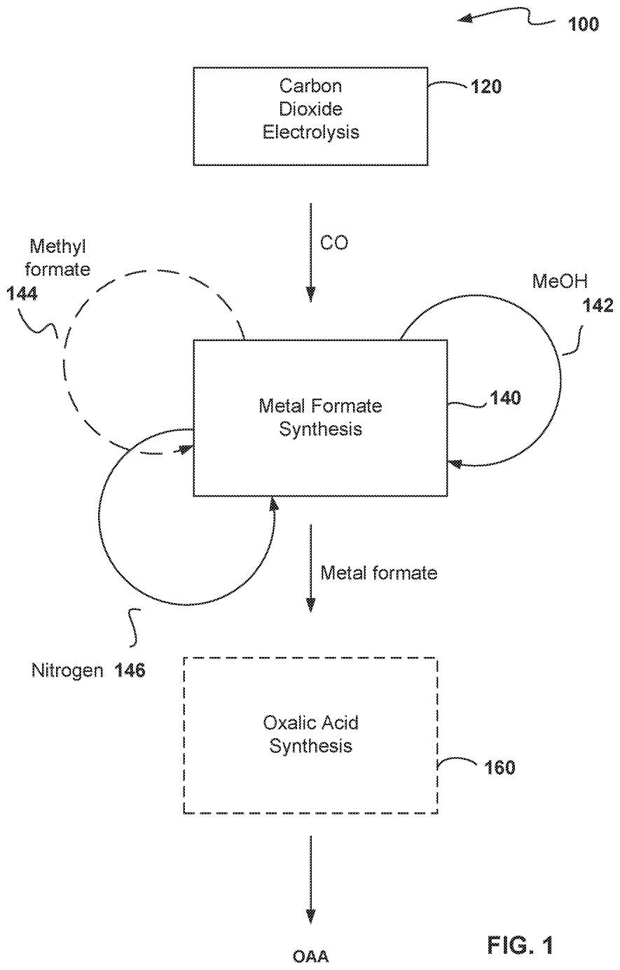

FIG. 1 is a schematic flow chart of certain embodiments of the invention; illustrating an operation 100, wherein a product gas from a carbon dioxide reactor 120 of the disclosure such as carbon monoxide can be used in one or more downstream processes, including metal formate synthesis 140. Optionally, the metal formate produced can further be used as a reactant in an oxalic acid synthesis process 160 to form oxalic acid. In some embodiments, operation 100 includes recycling of up to three chemical components during metal formate production. The system is configured to permit recycling of methanol 142; recycling of a carrier gas such as nitrogen 146, and optionally also recycling of methyl formate 144 (if it is present). Such a configuration results in a more efficient and environmentally-friendly production process.

### 2. The Electrolyzer

In some embodiments, process 100 begins with carbon dioxide electrolysis 120. In some embodiments, the electrolysis system 120 can include a carbon dioxide reactor, such as a reactor that generates carbon-containing products (e.g., CO, alkanes, alcohols, etc.) and/or hydrogen from an input (e.g., an input stream, such as a fluid stream) that includes carbon dioxide. The reactor may be configured to accept a gas-phase carbon dioxide input and/or performs the reaction(s) using gas-phase carbon dioxide (e.g., is a gas-phase reactor), but can additionally or alternatively accept liquid-phase carbon dioxide, supercritical fluid-phase carbon dioxide, solid-phase carbon dioxide, and/or any other suitable carbon dioxide input.

The electrolyzer may include one or more: electrodes (e.g., anode, cathode), catalysts (e.g., within and/or adjacent the cathode and/or anode), gas diffusion layers (e.g., adjacent the cathode and/or anode), and/or flow fields (e.g., defined within and/or adjacent the electrodes and/or gas diffusion layers, such as one or more channels defined opposing the cathode across the gas diffusion layer). In some embodiments, the electrolyzer includes a membrane stack or membrane electrode assembly (MEA) having one or more polymer electrolyte membranes (PEMs), providing ionic communication between the anode and cathode of the electrolyzer. In certain embodiments, the electrolyzer includes a membrane stack including: a cathode layer including a reduction catalyst and an ion-conducting polymer; a PEM membrane (e.g., bipolar membrane, monopolar membrane, etc.; membrane including one or more anion conductors such as anion exchange membranes (AEMs), proton and/or cation conductors such as proton exchange membranes, and/or any other suitable ion-conducting polymers; membrane including one or more buffer layers; etc.); and an anode layer including an oxidation catalyst and an ion-conducting polymer. The ion-conducting polymers of each layer can be the same or different ion-conducting polymers.

In some embodiments, one or more of the catalysts (e.g., reduction catalyst, oxidation catalyst) can include catalyst particles (e.g., defining a porous network of particles), such as nanoparticles. One or more of the catalysts can additionally or alternatively include one or more polymer electrolytes, optionally wherein the polymer electrolyte is mixed with the catalyst nanoparticles (e.g., arranged within the porous network, such as loaded into the open regions defined by the porous network). The catalyst nanoparticles can define one or more characteristic sizes (e.g., mean size, median size, minimum size, maximum size, size at a particular percentile of the particle size distribution, etc.), and/or the porous network can define a porosity (e.g., fraction of empty space within the network), density, circuitousness (e.g., characteristic path length per layer thickness, area, and/or volume, such as path through the empty spaces or path along interconnected particles, etc.), and/or any other suitable porous network metrics.

In some configurations, a bipolar MEA has the following stacked arrangement: cathode layer/cathode buffer layer (an anion conducting layer)/cation conductive layer (with may be a PEM)/anode layer. In some implementations, the bipolar MEA has a cathode layer containing an anion conductive polymer and/or an anode layer containing a cation conductive layer. In some implementations, the bipolar MEA has an anode buffer layer, which may contain a cation conductive material, between the cation conductive layer and the anode layer.

In some configurations, a bipolar MEA has the following stacked arrangement: cathode layer/cation conducting layer (with may be a PEM)/anion conductive layer/anode layer. In some applications, a bipolar MEA having this arrangement is configured in a system for reducing a carbonate and/or bicarbonate feedstock such as an aqueous solution of carbonate and/or bicarbonate.

In some configurations, an MEA has the following stacked arrangement: cathode layer/anion conducting layer/anode layer. In some implementations, this MEA has no cation conductive layers between the cathode layer and the anode layer. In some applications, an MEA containing only anion conductive material between the cathode and anode is configured in a system for reducing carbon monoxide feedstock.

In one example (“electrolyzer configuration A”), the system includes: a carbon fiber paper gas diffusion layer (e.g., Sigracet 39BC); a catalyst layer including approximately 20% by weight of approximately 4 nm gold particles on Vulcan carbon and an anion-conducting polymer (e.g., Fumasep FAA-3); a bipolar PEM; and a flow field such as a single, double, triple, or quadruple serpentine flow field or an interdigitated flow field. In a specific example, the electrodes define an area of approximately 25 cm², but can additionally or alternatively define any other suitable area.

In some embodiments, the electrolyzer includes one or more elements such as described in U.S. patent application Ser. No. 15/586,182, filed 3 May 2017, (U.S. Pat. No. 10,822,709), and titled “Reactor with Advanced Architecture for the Electrochemical Reaction of CO₂, CO and Other Chemical Compounds”, which is hereby incorporated in its entirety by this reference. However, the electrolyzer can additionally or alternatively include any other suitable elements in any suitable arrangement.

Additional information regarding optional embodiments and/or elements of the system and/or method are provided below, in US Patent Application Publication No. 2017/0321334, filed May 3, 2017, (U.S. Pat. No. 10,822,709), and in U.S. Provisional Patent Application No. 62/939,960, (U.S. Patent Pub. No. 2021-0207275) filed Nov. 25, 2019, which are incorporated herein by reference in their entireties.

A carbon oxide reduction electrolyzer may comprise more than one cells or MEAs. The multiple cells or MEAs may be arranged in a stack, electrically connected to one another in series and/or parallel. Unless otherwise indicated, all references herein to a carbon oxide reduction electrolyzer, a carbon oxide electrolyzer, and the like embody single cell electrolyzers and multicell stacks of electrolyzers.

A carbon oxide reduction electrolyzer may obtain carbon oxides from various sources. As mentioned, examples of carbon oxide reactants include carbon dioxide, carbon monoxide, carbonate, and/or bicarbonate. In certain embodiments, a carbonate or bicarbonate is provided in the form of an aqueous solution (e.g., an aqueous solution of potassium bicarbonate) that can be delivered to the cathode of a reduction cell. Carbonates and bicarbonates may be obtained from various sources (e.g., minerals) and/or by various reactions (e.g., reacting carbon dioxide with hydroxide).

A system may optionally include an upstream source of carbon dioxide input, connected to an input of a carbon dioxide electrolyzer of the disclosure, including one or more of: a biogas production system; an ethanol fermentation system such as corn ethanol production system, a beer production system, a wine production system; a natural gas processing system; a cement production system; a blast furnace system, for example a steel blast furnace system, capable of producing blast furnace gas; a coke gas production system; power plant systems, such as petroleum power plant systems, natural gas power plant systems, coal power plant systems; petroleum refinery systems; ethylene production systems; ammonia production systems; hydrogen production systems, such as water-gas shift systems; natural gas processing plants (e.g., Benfield processing); ethylene oxide production systems; aluminum smelting systems; liquified natural gas (LNG) production systems; solid feedstock gasifiers (e.g., municipal solid waste, biomass, or coal feedstocks); reformers (e.g., steam methane reformers, autothermal reformers); systems performing Boudouard reactions; direct air capture (DAC) of carbon dioxide process; and/or any other system capable of producing carbon dioxide. An upstream source of carbon dioxide may be connected directly to an input of a carbon dioxide reactor of the disclosure (e.g., serves as the input, such as connected to the reduction catalyst via the cathode flow field and/or gas diffusion layer, etc.) or alternatively the upstream source may be connected to a purification system; a gas compression system; or both a purification system and a gas compression system, in either order; which then connect to an input of a carbon dioxide system of the disclosure. Multiple purification and/or gas compression systems (e.g., scrubbers, etc.) may be employed.

The carbon dioxide, carbon monoxide, or carbonate provided as input to a carbon oxide reduction electrolyzer may, depending on the construction and operating conditions of the reactor, have a range of concentrations. In certain embodiments, carbon dioxide provided to a carbon dioxide reduction electrolyzer has a concentration of at least about 20 mole percent, or at least about 40 mole percent, or at least about 75 mole percent, or at least about 90 mole percent. In certain embodiments, carbon dioxide provided to a carbon dioxide reduction electrolyzer has a concentration of about 40 to 60 mole percent.

An upstream source of water for an electrolytic carbon oxide reduction electrolyzer may come from any of various source and in various forms such as purified tap water, purified sea water, a byproduct of direct air capture of water, optionally with capture of carbon dioxide, combustion processes that may also produce carbon dioxide feedstock, fuel cell byproduct, and the like.

A system may include an input of a downstream system, capable of transforming chemical outputs from a carbon dioxide electrolyzer of the disclosure, connected to an output of a carbon dioxide electrolyzer of the disclosure. A carbon dioxide electrolyzer output of the disclosure may be directly connected (e.g., via the cathode flow field and/or gas diffusion layer) to a downstream system, and/or the carbon dioxide electrolyzer output may be connected to a purification system; a gas compression system; or both a purification system and a gas compression system, in either order; which then optionally connect to an input of a downstream system. Multiple purification systems and/or gas compression systems may be employed.

A downstream system may produce carbon dioxide output in addition to other product outputs. A system may further include a connection between a carbon dioxide containing output of a downstream system and an input of a carbon dioxide electrolyzer. The carbon dioxide containing output of a downstream system may be directly connected to an input of a carbon dioxide reactor or alternatively the downstream carbon dioxide containing output may be connected to a purification system; a gas compression system; or both a purification system and a gas compression system, in either order; which then connect to an input of a carbon dioxide electrolyzer of the disclosure. Multiple purification systems and/or gas compression systems may be employed.

A carbon dioxide electrolyzer can make a range of products (for example, methane, ethylene, carbon monoxide (CO), molecular hydrogen (H₂), ethanol, formate, formic acid, acetate, acetic acid, propanol, butanol, ethane, methanol) that can be used in downstream systems and processes. Different carbon dioxide electrolyzers (e.g., including different layer stacks, catalysts and/or catalyst layers, PEMs, flow fields, gas diffusion layers, cell compression configurations, and/or any other suitable aspects, etc.) can be used to achieve different reduction products (e.g., product compositions such as HCR); however, different reduction products can additionally or alternatively be achieved by adjusting the operation parameters, and/or be otherwise achieved. Many possible downstream systems and processes release CO₂ (examples include bio-utilization of methane, bio-utilization of formic acid or formate, bio-utilization of acetic acid or acetate, Fischer-Tropsch processes, and methanol synthesis). A carbon dioxide recycling system sized appropriately for the specific application can be used in many of these cases to return CO₂ from the downstream system output to an input of a carbon dioxide electrolyzer of the disclosure to increase the carbon efficiency of the overall process.

A system may further include a source of electrical energy connected to a carbon dioxide electrolyzer, the source of electrical energy comprising one or more of: a solar electrical energy production system; a wind electrical energy production system; a geothermal electrical energy production system; a fossil fuel electrical energy production system; or any other system capable of electrical energy production.

A system may be employed to store electrical energy in the form of chemical energy. For example, power producers may produce excess power during off-peak usage periods. Systems containing carbon oxide reduction electrolyzers are able to respond quickly to a need to consume excess power. They do not need to warm up to operate, and they can be cycled between power on and power off states without deterioration of carbon dioxide reactors. The ability to respond quickly to power utilization needs allows systems to work well with intermittent sources of power such as solar electrical energy production systems, and wind electrical energy production systems.

An embodiment of a system may include an upstream bioreactor, a carbon dioxide electrolyzer, and an intermittent source of electrical energy. When electrical power is available from solar, or wind, or low off-peak demand, or other sources, a power availability detector may be used to start the carbon dioxide electrolyzer. In addition, the system may boost the output of the upstream bioreactor by, for example, raising the temperature of the upstream bioreactor and increasing the flow of nutrients to the upstream bioreactor. For other upstream carbon dioxide sources, other means may be used as necessary to increase the flow of carbon dioxide to an input of a carbon dioxide electrolyzer of the disclosure.

Any of the systems disclosed herein may include components (e.g., sensors, systems, etc.) to measure conditions, outputs, and inputs in the systems connected to a carbon dioxide electrolyzer. Such components may include chemical property measurement systems such as gas chromatographs, mass spectrometers, infrared spectrometers, visible light spectrometers, and/or ultraviolet light spectrometers; temperature detectors; flow rate measurement sensors; electrical power availability detectors; and/or any other monitoring systems. The monitoring systems can monitor the parameters of the input and/or output streams, the parameters of a component of the input and/or output streams (e.g., the impurity concentration, the carbon dioxide concentration, the product concentration, etc.), and/or monitor any other suitable parameter(s) of the stream.

Any of the systems disclosed herein may include components for responding to conditions measured in systems connected to a carbon dioxide reactor. Such components may include systems for adjusting flow rates, temperatures, power consumption or other system parameters. A system may include one or more carbon dioxide electrolyzer. However, the system can additionally or alternatively include any other suitable elements in any suitable arrangement. In various embodiments, one or more monitoring or sensing components is used in conjunction with a control system including a controller with appropriately programmed or constructed logic (e.g., processors and memory) for determining that one or more operating conditions should be modified and causing such operating condition(s) to be modified. Feedforward and/or feedback control systems may be employed.

### 2A. Methods for Electrolyzer Use

The method may be implemented using any of the components described above including an electrochemical carbon oxide reduction electrolyzer but can additionally or alternatively be implemented using any other suitable system(s). The method optionally includes running the electrolyzer under controlled process conditions (e.g., as described below in further detail) to produce the desired outputs (e.g., CO, H₂, etc.) in the desired ratios (e.g., molecular hydrogen-to-CCP ratio (HCR) and/or CCP-to-molecular hydrogen ratio), and/or altering the process conditions to alter the outputs and/or output ratios.

Running the electrolyzer can include: providing one or more inputs (e.g., gasses, liquids, solids, etc.), such as carbon dioxide, carbon monoxide, a carbon oxide source (e.g., waste gas), and/or water; causing all or some of the inputs to undergo reactions (e.g., by applying a voltage across the device electrodes), thereby generating products; and/or removing the products from the electrolyzer (e.g., as an output gas stream). Such reactions can include, for example, reducing carbon dioxide and/or water to generate products such as CO (and/or other CCPs, such as formic acid, methanol, glyoxal, methane, acetic acid, glycolaldehyde, ethylene glycol, acetaldehyde, ethanol, ethylene, hydroxyacetone, acetone, allyl alcohol, propionaldehyde, n-propanol, etc.), and/or H₂. However, running the electrolyzer can additionally or alternatively include causing any other suitable reactions to occur, and/or can additionally or alternatively include any other suitable elements performed in any suitable manner.

The method can include controlling the system to achieve a desired set of process conditions (e.g., aspects), such as process conditions known to result in a desired output metric value (e.g., a desired CCP:H₂ ratio, such as a CO:H₂ ratio). The method can additionally or alternatively include altering process conditions, such as based on a difference between actual and desired outputs (e.g., to reduce or eliminate the difference). For example, the method can include: imposing an initial set of process conditions; monitoring one or more output metrics (e.g., CCP:H₂ ratio); determining that an output metric differs from a target output metric (e.g., is greater than or less than the target); altering one or more process conditions to reduce the output metric difference (e.g., reducing or increasing a process condition value, such as a condition for which the output metric tends to increase or decrease along with an increasing process condition value); and optionally continuing to monitor the output metrics and/or alter the process conditions (e.g., implementing a closed-loop control of the process conditions based on the output metrics).

The method can optionally include determining the target output metric(s), which functions to determine which parameter(s) or aspect(s) to target (e.g., key parameter for a given application or downstream system). One or more target output metrics can be selected for a given process. The target output metric can be: the output metric associated with (e.g., predetermined for, dictated by, etc.) an application (e.g., applications described above, such as Fischer-Tropsch); randomly selected; empirically determined (e.g., through iterative testing and monitoring of downstream application performance); optimized (e.g., based on downstream application operation parameters, reactor operation parameters, etc.); specified by a user; and/or otherwise determined.

The method can optionally include determining the target value for the target output metric, which functions to identify a value (from a range of values) to target. In some variations, the target value can be a maximum or minimum value (e.g., maximum or minimum practically achievable value, theoretical maximum or minimum, etc.). However, the target value can additionally or alternatively not be an extremal value (e.g., can be an intermediate value or range of values between the maximum and minimum). The target value can be: a value associated with the application (e.g., predetermined, pre-associated); randomly selected; empirically determined (e.g., through iterative target value selection, monitoring of downstream application performance, and target value adjustment based on the application performance); optimized (e.g., based on downstream application operation parameters, reactor operation parameters, etc.); or otherwise determined. However, the target value can be any other suitable value and can be determined in any suitable manner.

Under some conditions, the method may achieve carbon dioxide conversion (e.g., CO fractional yield) greater than 95% (e.g., up to 100%), such as wherein the system, run under such conditions, can achieve at least the threshold conversion metric. However, the method can additionally or alternatively include achieving carbon dioxide conversion greater than 50%, 60%, 70%, 80%, 90%; between 10%-100%, such as 10-40, 30-50, 40-60, 50-70, 60-75, 70-85, 80-95, 90-95, 92-98, and/or 95-100%; and/or any other suitable carbon dioxide conversion.

The method optionally includes providing the electrolyzer products (or a subset thereof) to a downstream consumer of the products (e.g., as described above, such as regarding applications of the electrolyzer output; as described below, such as in the example section; etc.). The method can optionally include altering the electrolyzer products after they are produced (e.g., before feeding the altered products to a downstream consumer, etc.). Altering the electrolyzer products can optionally include purifying the products (e.g., removing impurities, such as SOₓ and/or NOₓ, from a reactor output stream). Altering the electrolyzer products can additionally or alternatively include mixing additional gasses (and/or other substances) into an electrolyzer output stream (and/or input stream), such as to achieve a desired output metric. In one variation, if the CO:H₂ ratio of the electrolyzer output differs from a desired value, the ratio can be adjusted by mixing the electrolyzer output with other gasses (e.g., substantially pure CO and/or H₂; another mixture of CO and H₂, such as previously produced and stored outputs of the electrolyzer, the output of a second electrolyzer, outputs and/or waste gasses of other systems, etc.). For example, the CO:H₂ ratio of the output stream (and/or gasses in any other portion of the electrolyzer) can be monitored (e.g., continuously during electrolyzer production), and deviations from the desired value can be compensated for by mixing in other gasses (e.g., adding CO and/or a CO-rich mixture to increase the ratio, adding H₂ and/or an H₂-rich mixture to decrease the ratio). This example may also include altering the process conditions in order to correct the electrolyzer outputs (e.g., as described above regarding closed-loop control). In a second variation, in which an external gas supply (e.g., the outputs and/or waste gasses of one or more other system, such as a steel mill) is fed to a downstream consumer (e.g., a gas fermenter), the electrolyzer products are used to alter the CCP:H₂ ratio (e.g., CO:H₂ ratio) of the external gas supply (e.g., if the CCP:H₂ ratio of the external gas supply differs from a desired value, mixing in the electrolyzer products to achieve the desired value). For example, based on the deviation of the external gas supply from the desired value, the process conditions can be controlled to alter the CO:H₂ ratio of the electrolyzer products (e.g., increasing the ratio in response to a CO-poor external gas supply, decreasing the ratio in response to a CO-rich external gas supply), and/or the quantity of electrolyzer product mixed into the external gas supply can be controlled (e.g., to achieve the desired value). However, the electrolyzer output stream can additionally or alternatively be altered in any other suitable manner or can be used without alteration.

In some examples, the method includes determining one or more metrics (e.g., operation metrics) associated with the one or more upstream and/or downstream elements of the system (e.g., downstream reactors, upstream inputs, etc.). Such operation metrics can include, for example: electrolyzer conditions such as temperature, pressure, etc.; downstream reactor and/or upstream source output metrics such as output quantity, composition, purity, etc.; metrics associated with other inputs for the downstream reactor(s), such as input quantity, composition, purity, etc.; reactor efficiency metrics; and/or any other suitable metrics. In such examples, the method may include altering carbon dioxide electrolyzer operation based on the metrics (e.g., to improve and/or maintain operation of the downstream reactor; to improve and/or maintain operation of the carbon dioxide electrolyzer, such as to accommodate changes in the upstream source; to improve and/or maintain a target output metric, such as HCR or reduction product concentration, such as given a varying carbon dioxide source; etc.), such as by altering the HCR of the carbon dioxide reactor output. However, the method can additionally or alternatively include determining any other suitable metrics and/or acting (e.g., based on the metrics) in any other suitable manner.

### 2B. Electrolyzer Process Conditions

The process conditions can include, e.g., input carbon dioxide flow rate and/or pressure, input gas hydration, current density, voltage (e.g., maintained between about 1.5 V and 3 V, additionally or alternatively operated at less than about 1.5 V, between about 2 V-2.5 V, between about 2 V-4 V, greater than about 4 V, and/or at any other suitable voltage(s)), and/or temperature. The process conditions can additionally or alternatively include system configurations, such as gas diffusion layer aspects, catalyst aspects, flow field aspects, and/or PEM aspects. However, any other suitable process condition can be controlled or targeted. The process condition can be uncontrolled (e.g., dictated by an upstream system), controlled to meet a target value (e.g., wherein the target value can be determined based on the application receiving the reactor output, the instantaneous or anticipated reactor operation parameters, or otherwise determined), or otherwise determined.

The process conditions may include a pressure (e.g., input gas pressure, electrolyzer pressure, etc.) greater than atmospheric pressure (e.g., within and/or greater than a threshold pressure range, such as about 1-5, about 5-10, about 10-20, about 20-50, about 50-100, about 100-300, about 300-1000, about 1-10, about 5-50, about 10-100, about 20-500, and/or greater than about 1000 atm, about 14-50, about 50-150, about 100-300, about 200-500, about 500-1000, about 750-1500, about 1000-3000, about 3000-10,000, about 10,000-20,000, and/or greater than about 20,000 psi, etc.) and/or greater than pressures typically feasible in electrolyzers other than gas-phase electrolyzers, but can additionally or alternatively include pressures substantially equal to 1 atmosphere, less than about 1 atmosphere, and/or any other suitable pressures. The process conditions may include a temperature (e.g., reactor temperature) greater than typical room temperature (e.g., within and/or greater than a threshold temperature range, such as about 25-50, about 40-60, about 50-100, about 50-75, about 70-100, and/or greater than about 100° C., etc.) and/or greater than temperatures typically feasible in electrolyzers other than gas-phase electrolyzers, but can additionally or alternatively include temperatures substantially equal to room temperature (e.g., about 20-30° C.), less than room temperature, and/or any other suitable temperatures. However, the process conditions can additionally or alternatively include any other suitable process conditions.

A higher carbon dioxide flow rate can lead to increased production of CCPs such as CO (e.g., due to greater availability of carbon dioxide for reduction), and thus an increased CCP:H₂ ratio (and correspondingly, lower carbon dioxide flow rate can lead to decreased CCP production and CCP:H₂ ratio). In some embodiments, higher carbon dioxide flow rate can also result in reduced carbon dioxide conversion efficiency, thereby diluting the output stream (e.g., syngas output) with unreacted carbon dioxide. For example, carbon dioxide flow rate (e.g., measured at the reactor inlet) can be maintained at one or more values in the range of about 0.1-1000 sccm/cm² (e.g., about 0.1-1, about 1-10, about 10-100, and/or about 100-1000 sccm/cm²).

In a first specific example of control based on input gas flow rate, electrolyzer configuration A with a triple serpentine flow field is used, electrolyzer pressure is substantially maintained at 120 psi, current density is substantially maintained at 500 mA/cm², and electrolyzer temperature is substantially maintained at 30° C. In this specific example, substantially pure carbon dioxide gas is input at various flow rates, wherein input flow rates (e.g., measured at the reactor inlet) of 12 sccm/cm², 20 sccm/cm², and 40 sccm/cm² result in CO:H₂ ratios of approximately 1:1, 2:1.1, and 4:1, respectively.

In a second specific example of control based on input gas flow rate, electrolyzer configuration A with a serpentine flow field is used, electrolyzer pressure is substantially maintained at 130 psi, and current density is substantially maintained at 500 mA/cm². In this specific example, substantially pure carbon dioxide gas input at a 40 sccm/cm² flow rate results in a CO:H₂ ratio of approximately 8:2, whereas a 12 sccm/cm² flow rate results in an approximately 1:1 ratio.

Higher carbon dioxide pressure can lead to increased CCP fractional yield and/or CCP:H₂ ratio (and correspondingly, lower carbon dioxide pressure can lead to decreased CCP fractional yield and/or CCP:H₂ ratio). First, increased carbon dioxide pressure can result in greater availability of carbon dioxide for reduction, thereby increasing the total production of CCPs. Second, higher pressure at the catalyst can reduce water ingress to the catalyst (e.g., from the cathode), thereby lowering the amount of water available for reduction, which can directly increase the CCP:H₂ ratio and/or can reduce competition for catalyst reaction sites and/or reaction energy (e.g., thereby favoring reduction of carbon dioxide). Thus, in some embodiments (e.g., in which high CCP fractional yield and/or CCP:H₂ ratio is desired), high electrolyzer pressure (e.g., greater than 100 psi, up to but no greater than a carbon dioxide phase transition pressure, such as a critical pressure of 1070 psi, etc.) may be employed. For example, electrolyzer pressure can be maintained at one or more values in the range of about 1-1100 psi (e.g., about 1-10, about 10-100, about 100-300, about 200-600, and/or about 500-1100 psi), and/or at any other suitable pressure.

In a specific example of control based on electrolyzer pressure, electrolyzer configuration A with a single serpentine flow field is used, substantially pure carbon dioxide gas is input at about 100 sccm/cm², current density is substantially maintained at about 150 mA/cm², and electrolyzer temperature is substantially maintained at about 20° C. In this specific example, electrolyzer pressure is substantially maintained at various pressures, wherein electrolyzer pressures of 25, 50, 75, and 100 psi result in CO:H₂ ratios of approximately 3:2, 2.4:1, 3:1, and 5:1 and CO fractional yields of approximately 59%, 69%, 75%, and 84%, respectively.

Increasing input gas hydration can lead to increased water reduction (e.g., due to greater availability of water for reduction), and thus to a decreased CCP:H₂ ratio. For a substantially pure carbon dioxide input, only small amounts of water reach the catalyst (coming almost exclusively from the cathode side of the reactor), leading to a higher CCP:H₂ ratio. In contrast, when hydrated input gas is used, significant amounts of water from the input gas can reach the catalyst and react. For example, input gas hydration (e.g., proportion of water vapor in the input gas) can be maintained at one or more values in the range of 0% (e.g., substantially pure carbon dioxide, substantially unhydrated input gas) to 100% (e.g., 0-1, 1-3, 3-5, 5-7, 7-10, 10-15, 15-25, 25-50, 50-75, and/or 75-100 percent).

In a specific example of control based on input gas hydration, electrolyzer configuration A with a single serpentine flow field is used, current density is substantially maintained at 50 mA/cm², electrolyzer pressure is substantially maintained at 12 psi, and electrolyzer temperature is substantially maintained at 20° C. In this specific example, carbon dioxide gas with varying amounts of hydration is input at 100 sccm/cm², wherein pure carbon dioxide input gas results in a CO:H₂ ratio of approximately 3:2, input gas with 12.2% hydration results in a CO:H₂ ratio of approximately 1:5.67, and intermediate hydration amounts result in CO:H₂ ratios between these two values.

Electrolyzers can exhibit different regimes of CCP and H₂ production with respect to current density. In an idealized electrolyzer, at low current densities, no water reduction occurs and all current goes to reducing carbon dioxide, resulting in a substantially linear dependence of CO production on current and substantially no H₂ production; whereas at higher current densities, additional current (e.g., above a threshold current at which substantially all carbon dioxide is already being consumed) is used to reduce water, resulting in a substantially linear dependence of H₂ production on the additional current and substantially constant CO production. In many typical electrolyzer, these idealities are loosened, but the two general regimes are still exhibited: CO production increases much faster than H₂ production in the low current density regime, then approaches a plateau in the higher current density regime while H₂ production increases more rapidly. The method can include controlling CO and/or H₂ production (e.g., controlling CO:H₂ ratio) by operating at any or all of a wide range of current densities (e.g., controlling the electrolyzer operation within the low and/or high current density regime, etc.). In some embodiments, the use of gas phase input carbon dioxide can enable relatively high current densities (whereas electrolyzers using aqueous carbon dioxide may be limited to current densities of tens of mA/cm² or less). For example, the method can include operating at current densities between about 1 mA/cm² and 100 A/cm² (e.g., about 1-75 mA/cm², about 50-100 mA/cm², about 100-200 mA/cm², about 200-500 mA/cm², about 500-1000 mA/cm², about 50-1000 mA/cm², about 0.5-10 A/cm², about 1-2 A/cm², about 2-5 A/cm², about 5-10 A/cm², about 5-100 A/cm², about 10-20 A/cm², about 20-50 A/cm², about 50-100 A/cm², etc.; at, above, or below a threshold value such as about 50 mA/cm², about 65 mA/cm², about 80 mA/cm², about 90 mA/cm², about 100 mA/cm², about 110 mA/cm², about 120 mA/cm², about 130 mA/cm², about 140 mA/cm², about 150 mA/cm², about 200 mA/cm², about 300 mA/cm², about 500 mA/cm², about 700 mA/cm², about 1000 mA/cm², about 1500 mA/cm², etc.) and/or at any other suitable current densities.

In some embodiments, increased electrolyzer temperature can result in a reduced CO:H₂ ratio (e.g., due to increased ingress of water from the cathode, increased reactivity of water, etc.). The method can include controlling electrolyzer temperature within an operation range, such as a range between a minimum temperature (e.g., a water freezing temperature such as 0° C.) and a maximum temperature (e.g., about 40° C., about 50° C., about 60° C., about 75° C., etc.; a water boiling temperature such as 100° C.), in order to control CO:H₂ ratio and/or any other suitable output metrics.

In a specific example of control based on electrolyzer temperature, electrolyzer configuration A with a quadruple serpentine flow field is used, substantially pure carbon dioxide gas is input at 70 sccm/cm², current density is substantially maintained at 150 mA/cm², and electrolyzer pressure is substantially maintained at 100 psi. In this specific example, electrolyzer temperature is substantially maintained at various temperatures, wherein electrolyzer temperatures of 26.7, 35, 38.7, and 41.9° C. result in CO:H₂ ratios of approximately 1:0.4, 2:1, 1:1.8, and 1:3, respectively.

Characteristics of the gas diffusion layer (GDL) can additionally or alternatively be used to affect CCP and/or H₂ production. For example, the GDL hydrophobicity can alter H₂ production (e.g., by affecting water transport), wherein a more hydrophilic GDL favors H₂ production (thereby reducing the CCP:H₂ ratio) and a more hydrophobic GDL inhibits H₂ production (thereby increasing the CCP:H₂ ratio). Other GDL characteristics, such as thickness and/or pore size, can also be used to alter the reactor output.

Characteristics of the membrane (e.g., polymer electrolyte membrane) can additionally or alternatively be used to affect CCP and/or H₂ production. In examples, an anion exchange membrane, which favors CCP production, can be used to achieve high CCP: H₂ ratios, a cation exchange membrane, which favors H₂ production, can be used to achieve low CCP:H₂ ratios, and hybrid membranes (e.g., enabling both anion and cation transport) exhibiting various anion and cation transport characteristics (e.g., mobilities) can be used to achieve various intermediate ratios (e.g., membranes favoring anion transport for higher ratios, membranes favoring cation transport for lower ratios).

Characteristics of the catalysts (e.g., particle size, catalyst species, etc.) can additionally or alternatively be used to affect CCP and/or H₂ production. For example, larger catalyst particles can result in poor carbon dioxide transport, thereby inhibiting CCP production and reducing the CCP:H₂ ratio, whereas smaller catalyst particles can favor CCP production, thereby increasing the ratio. The relative number of active sites with high turnover frequency for hydrogen evolution (“hydrogen sites”) and those with high turnover frequency for carbon dioxide reduction (“carbon dioxide sites”) can additionally or alternatively be dependent on catalyst particle size: larger catalyst particles typically have a higher ratio of hydrogen sites to carbon dioxide sites, favoring H₂ production, whereas smaller catalyst particles typically have a lower ratio, favoring CO production. The catalyst type (e.g., catalyst species) can additionally or alternatively be used to control the electrolyzer output, such as by employing a mixture of one or more catalyst materials, wherein a first set of catalyst materials (e.g., gold) favor carbon dioxide reduction and a second set of catalyst materials (e.g., platinum) favor water reduction. In examples, a substantially pure gold catalyst can be used to achieve high CCP:H₂ ratios, a substantially pure platinum catalyst can be used to achieve low CCP:H₂ ratios, and gold-platinum mixtures (e.g., alloyed particles, mixtures of gold particles and platinum particles, etc.) of varying composition can be used to achieve various intermediate ratios (e.g., more gold for higher ratios, more platinum for lower ratios). The catalyst can additionally or alternatively include V, Cr, Mn, Fe, Co, Ni, Cu, Sn, Zr, Nb, Mo, Ru, Rh, Pd, Ag, Cd, Hf, Ta, W, Re, Ir, Hg, Al, Si, In, Ga, Tl, Pb, Bi, Sb, Te, Sm, Tb, Ce, Nd, and/or combinations thereof. The catalyst can additionally or alternatively be associated with (e.g., attached to, supported by, embedded in, adjacent, in contact with, etc.) one or more support structures (e.g., support particles, support matrix, etc.), which may be conductive support structures such as carbon, boron-doped diamond, and/or fluorine-doped tin oxide. However, the catalyst can additionally or alternatively include any other suitable materials.

In a specific example of control based on catalyst particle size, variations of electrolyzer configuration A with two catalyst particle sizes are used, both with electrolyzer temperature substantially maintained at 30° C., electrolyzer pressure substantially maintained at 100 psi, an interdigitated flow field, substantially pure carbon dioxide gas input at 10 sccm/cm², and current density substantially maintained at 500 mA/cm². The first set of catalyst particles have a characteristic size of 4 nm (as in the standard reactor configuration A), resulting in an HCR of 1:1.6 and a voltage of 3.8 V. The second set of catalyst particles have a characteristic size of 20 nm, resulting in an HCR of 1:2.8 and a voltage of 4.2 V.

Characteristics of electrolyzer cell compression can additionally or alternatively be used to affect CCP and/or H₂ production. In a specific example of control based on electrolyzer cell compression, electrolyzer configuration A is used with two different gasket thicknesses (resulting in greater compression for a larger gasket thickness), both with electrolyzer temperature substantially maintained at 30° C., electrolyzer pressure substantially maintained at 100 psi, a triple serpentine flow field, substantially pure carbon dioxide gas input at 40 sccm/cm², and current density substantially maintained at 500 mA/cm². The first gasket is 0.012 inches thick, resulting in an HCR of 1:4 and a voltage of 3.6 V. The second gasket is 0.010 inches thick, resulting in an HCR of 1:10.1 and a voltage of 3.8 V.

Characteristics of the flow field can additionally or alternatively be used to affect CCP and/or H₂ production. In a first specific example of control based on flow field characteristics, electrolyzer configuration A is used under two different sets of process conditions, both with electrolyzer temperature substantially maintained at 30° C. and electrolyzer pressure substantially maintained at 120 psi. In the first set of conditions, an interdigitated flow field is used, substantially pure carbon dioxide gas is input at 10 sccm/cm², and current density is substantially maintained at 160 mA/cm², resulting in a CO:H₂ ratio of 1.6:1. In the second set of conditions, a quadruple serpentine flow field is used, substantially pure carbon dioxide gas is input at 40 sccm/cm², and current density is substantially maintained at 120 mA/cm², resulting in a CO:H₂ ratio of 18.5:1.

In a second specific example of control based on flow field characteristics, electrolyzer configuration A is used under two different sets of process conditions, both with electrolyzer temperature substantially maintained at 30° C., electrolyzer pressure substantially maintained at 100 psi, substantially pure carbon dioxide gas input at 40 sccm/cm², and current density is substantially maintained at 500 mA/cm². In the first set of conditions, an interdigitated flow field is used and a voltage of 3.6 V is substantially maintained, resulting in a CO:H₂ ratio of 1.6:1. In the second set of conditions, a triple serpentine flow field is used and a voltage of 3.8 V is substantially maintained, resulting in a CO:H₂ ratio of 10.1:1.

However, any other suitable flow field can additionally or alternatively be employed to control the electrolyzer outputs, the process conditions can additionally or alternatively include any other suitable electrolyzer conditions, and the method can additionally or alternatively include controlling the electrolyzer output in any suitable manner.

### 2C. Electrolyzer Impurity Tolerance

In some embodiments, such as embodiments in which the electrolyzer is run at a high pressure and/or the catalyst is held at low voltage (e.g., negative voltage relative to the anode), the system and/or method may achieve high tolerance to impurities and/or dilute carbon dioxide inputs (e.g., as compared to other carbon dioxide reactors), such as tolerance to poisoning by impurities in the electrolyzer input(s) and/or to inputs diluted by species such as methane, CO, O₂, and/or N₂. For example, the method can include determining target process conditions (e.g., electrolyzer configuration such as PEM type, high target electrolyzer pressure, etc.) to achieve impurity and/or dilute input tolerance (e.g., always selecting such process conditions; selecting such process conditions in response to a current and/or anticipated state of the reactor input, such as an impure and/or dilute state; etc.). These impurities can include species typically present in electrolyzer input streams (e.g., products of coal and/or natural gas combustion, such as outputs from coal- or natural gas-fired power plants), such as SOₓ and/or NOₓ, and/or can include any other impurities such as ammonia, hydrogen sulfide, and mercury. In one example, the system and/or method are capable of functioning effectively using input streams including up to 4% CO, 6% O₂, 10% N₂, 800 ppm NOₓ, and/or 100 ppm SOₓ, with a sum of CO, O₂, and N₂ impurities, e.g., no greater than 10%.

In a specific example of dilute input tolerance, electrolyzer configuration A with a single serpentine flow field is used, current density is substantially maintained at 160 mA/cm², electrolyzer pressure is substantially maintained at 110 psi, electrolyzer temperature is substantially maintained at 20° C., and carbon dioxide-containing gasses with various levels of dilution in methane or nitrogen are input at 200 sccm/cm². In this specific example, electrolyzer performance is highly tolerant of methane dilution up to at least 50% methane, wherein methane concentrations of 0%, 25%, and 50% result in CO:H₂ ratios between 9.5:1 and 8.5:1 and CO fractional yields greater than 90%. More significant performance reduction is observed using 75% methane, with a reduction in CO fractional yield to approximately 84%. In this specific example, similar tolerance to nitrogen dilution is observed, wherein nitrogen concentrations of 0%, 25%, 50%, and 75% result in CO:H₂ ratios between 9:1 and 8:1, and nitrogen concentrations up to 50% result in CO fractional yields greater than 85% (with 75% nitrogen concentration resulting in a CO fractional yield of approximately 81%).

In a specific example of impurity tolerance, electrolyzer configuration A with a single serpentine flow field is used, current density is substantially maintained at 150 mA/cm², electrolyzer pressure is substantially maintained at 100 psi, electrolyzer temperature is substantially maintained between 20° C. and 25° C., and carbon dioxide-containing gasses with various impurities are input at 100 sccm/cm². In this specific example, electrolyzer output metrics (e.g., CO fractional yield) under the various impurity conditions are compared to baseline electrolyzer performance under the same conditions but using a substantially impurity-free carbon dioxide input. In this specific example, electrolyzer performance was shown not to deviate significantly from the baseline performance for CO concentrations of 4% or less, for NOₓ concentrations of 800 ppm or less, for SOₓ concentrations of 120 ppm or less, or for oxygen concentrations of 6% or less.

However, the system and/or method can additionally or alternatively exhibit any suitable tolerance to impure and/or dilute inputs or exhibit no such tolerance.

In certain embodiments, an impurity or multiple impurities pass through the carbon oxide reduction electrolyzer to an output stream where they are (a) separated upstream of another chemical reactor, and/or (b) passed into another chemical reactor. In embodiments where impurities in an output stream are passed to another chemical reactor, impurities may be used by the other reactor in the chemical manipulation of that process. For example, hydrogen sulfide or other sulfur-containing impurity may be employed by microbial species in a downstream bioreactor.

### 2D. Electrolyzer Configuration Selection

One or more system configurations may be employed based on output HCR considerations, such as based on a desired output HCR (e.g., given a particular set of process conditions and/or a range of acceptable process conditions) and/or HCR range.

In some embodiments, this includes: at a first electrolyzer, such as a gas-phase electrolyzer, accepting an input including a carbon oxide and electrochemically producing a first reduction product (e.g., including molecular hydrogen and/or one or more CCPs other than the carbon oxide input at a first HCR) from the input (e.g., under a first set of process conditions). The choice of the first electrolyzer design and its operating conditions may include determining a desired HCR and/or HCR range (e.g., based on downstream reactor metrics, market price metrics, efficiency metrics, and/or any other suitable metrics) and selecting a system configuration (e.g., for a second electrolyzer) based on the first HCR and/or the desired HCR (e.g., such that the second electrolyzer will or can output a reduction product with an HCR closer to the desired HCR relative to the first HCR, optionally substantially under the first set of process conditions but additionally or alternatively under any other suitable process conditions). For example, the configuration for the second electrolyzer can be selected such that the second would, under conditions substantially identical to those of the first electrolyzer (e.g., while accepting the input under the first set of process conditions), produce a second reduction product from the input, wherein the second reduction product includes molecular hydrogen and the same CCSs as the first reduction product (e.g., includes substantially all species present in the first reduction product), wherein the second reduction product defines a second HCR substantially different from the first HCR, wherein the second HCR may be closer to the desired HCR than the first HCR. Substantial difference between the first HCR and second HCR, for this example and/or any other embodiment described herein, can include the second HCR: being closer to the desired HCR than the first HCR; differing from the first HCR (e.g., being greater or lesser than the first HCR) by at least 1%, 5%, 10%, 30%, 40%, 50%, 60%, 70%, 80%, 90%, 0.5-5%, 2-10%, 5-25%, 20-50%, 40-80%, and/or 75-100%; and/or otherwise differing from the first HCR.

In some embodiments, selecting system configurations can include selecting one or more aspects of a PEM, such as to alter the output HCR. Such selection can include selecting membrane compositions (e.g., different polymer species) and/or microstructures, selecting membrane layer thicknesses, and/or selecting any other suitable aspects of the PEM. In some examples, such selection includes selecting a thickness of an anion exchange membrane and/or proton exchange membrane (e.g., wherein a bipolar PEM with more AEM will tend to produce a lower output HCR than one with more proton exchange membrane). In a first specific example, selecting a thinner AEM (e.g., thinner than a reference AEM thickness such as a thickness of the first reactor AEM, thinner than an optimized AEM thickness substantially corresponding to optimal CCP production, etc.) can result in a reactor configured to produce a higher output HCR, whereas selecting a thicker AEM (e.g., thicker than the reference AEM thickness but optionally no thicker than the optimized AEM thickness) can result in a reactor configured to produce a lower output HCR.

Selecting system configurations can additionally or alternatively include selecting one or more aspects of electrolyzer catalyst(s) (e.g., reduction catalyst, oxidation catalyst), such as to alter the output HCR. In some variations, selecting electrolyzer catalyst aspects can include selecting a catalyst layer thickness (e.g., wherein a thicker reduction catalyst will tend to produce a higher HCR). In one example, selecting a thicker reduction catalyst layer (e.g., thicker than a reference reduction catalyst layer thickness such as a thickness of the first electrolyzer reduction catalyst layer, thicker than an optimized reduction catalyst layer thickness substantially corresponding to optimal CCP production, etc.) can result in an electrolyzer configured to produce a higher output HCR, whereas selecting a thinner reduction catalyst layer (e.g., thinner than the reference reduction catalyst layer thickness but optionally no thinner than the optimized reduction catalyst layer thickness) can result in an electrolyzer configured to produce a lower output HCR.

Selecting electrolyzer catalyst aspects can additionally or alternatively include (e.g., in embodiments in which a catalyst layer includes catalyst particles, such as nanoparticles, defining a porous network) selecting a catalyst porosity (e.g., wherein a more porous reduction catalyst network will tend to produce a lower HCR). In one example, selecting a less porous reduction catalyst network (e.g., less porous than a reference reduction catalyst such as a porosity of the first electrolyzer reduction catalyst network, less porous than an optimized reduction catalyst substantially corresponding to optimal CCP production, etc.) can result in an electrolyzer configured to produce a higher output HCR, whereas selecting a more porous reduction catalyst (e.g., more porous than the reference reduction catalyst but optionally no more porous than the optimized reduction catalyst) can result in an electrolyzer configured to produce a lower output HCR.

Selecting electrolyzer catalyst aspects can additionally or alternatively include (e.g., in embodiments in which a catalyst layer includes catalyst particles, such as nanoparticles, and one or more polymer electrolytes, such as wherein the catalyst particles define a porous network that contains the polymer electrolyte and/or are mixed into a medium including the polymer electrolyte) selecting a catalyst-to-polymer electrolyte ratio (CPR) (e.g., wherein a higher reduction catalyst CPR will tend to produce a higher HCR), such as by selecting a degree of polymer electrolyte loading into a porous reduction catalyst network. In one example, selecting a higher reduction catalyst CPR (e.g., higher CPR than a reference reduction catalyst CPR such as a CPR of the first electrolyzer reduction catalyst network, higher CPR than an optimized reduction catalyst substantially corresponding to optimal CCP production, etc.) can result in an electrolyzer configured to produce a higher output HCR, whereas selecting a lower CPR reduction catalyst (e.g., lower CPR than the reference reduction catalyst but optionally no lower than the optimized reduction catalyst CPR) can result in an electrolyzer configured to produce a lower output HCR.

Selecting electrolyzer catalyst aspects can additionally or alternatively include (e.g., in embodiments in which a catalyst layer includes catalyst particles, such as nanoparticles) selecting a characteristic catalyst particle size (e.g., wherein a larger particle size will tend to produce a higher HCR). In one example, selecting a larger reduction catalyst particle size (e.g., larger than the particles of a reference reduction catalyst such as the first electrolyzer reduction catalyst, larger than an optimized reduction catalyst substantially corresponding to optimal CCP production, etc.) can result in an electrolyzer configured to produce a higher output HCR, whereas selecting a smaller reduction catalyst particle size (e.g., smaller than the particles of the reference reduction catalyst but, e.g., no smaller than the particles of the optimized reduction catalyst) can result in an electrolyzer configured to produce a lower output HCR. However, the method can additionally or alternatively include selecting any other suitable electrolyzer catalyst aspects.

The method can additionally or alternatively include selecting an electrolyzer cell compression (e.g., wherein lower compression will tend to result in higher HCR and higher compression will tend to result in lower HCR), a flow field, and/or any other suitable aspects of the system.

International Publication No. WO/2022/031726, published on 10 Feb. 2022 is incorporated herein by reference in its entirety.

The electrolyzer design and operating conditions can be tuned for particular applications, and for producing a cathode output having specified compositions. In some implementations, one or more general principles may be applied to operate in a way that produces a required output stream composition.
1. Restrict carbon dioxide reactant availability at the cathode active sites and/or increase current density at the cathode. These operating condition ranges tend to produce the following results: (a) initially, upon decreasing the carbon dioxide reactant availability and/or increasing the current density, the fraction of CO₂ converted to CO increases (i.e., CO:CO₂ in the output stream increases); (b) at some point, upon further decreasing the carbon dioxide reactant availability and/or increasing the current density, the hydrogen ion reduction reaction becomes more pronounced (i.e., H₂:CO increases). Electrolyzers that can operate with relatively little carbon dioxide input/availability may have flow fields or gas diffusion components that restrict carbon dioxide from reaching active sites on the electrolyzer cathode. In certain embodiments, flow field designs that are not interdigitated, and such flow field designs that have long paths such as serpentine paths between the source of CO₂ and the cathode result in higher ratios of CO:H₂. Interdigitated flow field forces input gas (carbon oxide) to flow through the gas diffusion layer before exiting at a different location on the flow field. Non-interdigitated designs have long continuous paths for the carbon oxide feed gas to flow into and out of the cathode. Channels on the inlet side are spaced from the channels on the outlet side. In certain embodiments, gas diffusion electrodes that are relatively thick restrict CO₂ mass transport to the cathode active sites and therefor tend to increase the ratio of CO:CO₂ and/or H₂:CO.
2. Make hydrogen ions relatively more available at the cathode. Making hydrogen ions relatively more available at the cathode may produce a cathode product stream with a relatively high ratio of H₂:CO. Electrolyzers configured in a way that provide a relatively hydrogen rich product may employ designs that (a) starve the cathode of carbon dioxide reactant (as described in 1), (b) permit a relatively high flux of hydrogen ions to be transported from the anode, where they are generated, to the cathode, and/or (c) operate at a relatively high cell temperature. Electrolyzers that can operate with a relatively high flux of hydrogen ions to the cathode may have MEAs with cation conducting polymers and/or mixed ion conducting polymers at the cathode. Alternatively or additionally, in MEAs including a cathode buffer layer, the layer is designed to be relatively thin and/or have a relatively high hydrogen ion transference number.
3. Make hydrogen ions less available at the cathode. Making hydrogen ions relatively more less at the cathode may produce a cathode product stream with relatively high ratios of CO:H₂. Electrolyzers configured in a way that provides a relatively hydrogen poor product may employ designs that (a) provide the cathode with surplus carbon dioxide reactant for a given current density, (b) contain MEA designs that prevent hydrogen ions from reaching the cathode, and/or (c) operate at a relatively low cell temperature.

### 2E. Direct Air Capture of CO₂

In certain embodiments, an electrolytic carbon dioxide reduction system uses carbon dioxide received directly from air. A system for such embodiments includes a direct air CO₂ capture subsystem and a carbon dioxide reduction electrolyzer subsystem. The system is configured so that CO₂ from the capture subsystem supplies CO₂, directly or indirectly, to the cathode side of the electrolyzer subsystem.

Because air is often the only significant feedstock, an air capture CO₂ electrolysis system may be deployed at any location where there is space for the system components. In some deployments, the system occupies a relatively unpopulated area. In some deployments, the system occupies a populated area. In some embodiments, the system is deployed, at least partially, on a vehicle or vessel. For example, an air capture unit may be provided on a vehicle or vessel while a carbon dioxide electrolyzer may be provided at a port or offshore platform. In some cases, the deployment location has a ready supply of energy, e.g., a location where solar and/or wind power is plentiful. In some cases, the deployment location is a desert. In some embodiments, the system is deployed on large vessel such as a cargo ship or military vessel such as an aircraft carrier. In some embodiments, the energy source is provided by a solar or windfarm associated with an offshore platform or port, while carbon dioxide capture unit is provided on a ship or other watercraft. A carbon dioxide electrolyzer may be provided on the offshore platform or port.

The system may be designed so that air or other gas is provided under specified conditions to the CO₂ capture subsystem. In certain embodiments, fans, vacuum pumps, or simply wind are used to deliver air to a CO₂ capture subsystem.

In certain embodiments, the CO₂ capture subsystem comprises two stages: a first stage in which air is contacted with a sorbent that removes CO₂ from air (phase 1), and second stage in which heat, electricity, pressure, and/or humidity is applied to the sorbent to release CO₂ and/or water (phase 2).

In some implementations, the CO₂ capture subsystem employs a solid or liquid absorbent or adsorbent to capture the CO₂ in phase 1. In various implementations, phase 1 is performed at ambient conditions or near ambient conditions. In phase 2, a temperature, electrical, pressure, and/or moisture swing is applied, causing the absorbed or adsorbed CO₂, and optionally water, to be released.

In certain embodiments, the absorbent is heated to release the CO₂. As an example, the sorbent is heated from, e.g., ambient temperature (e.g., about 20-40° C.) to a temperature of at least about 75° C. to release CO₂ and optionally water. In some cases, the temperature swing is from ambient to about 50 to 1000° C. or from ambient to about 75-200° C. or from ambient to about 600 to 1000° C. As an example, the sorbent is heated for a duration of time sufficient remove a desired fraction of CO₂ and optionally water. The duration is a function of the amount of the sorbent to be treated, the fraction of CO₂ and/or water to be removed, and the heat transfer to the sorbent.

In certain embodiments, the absorbent is exposed to humidity to release the CO₂. As an example, the sorbent is initially exposed to dry air (e.g., air having at most about 50 mole % water, or at most about 30 mole % water, or at most about 5 mole % water) and subsequently exposed to humid vapor (e.g., air having at least about 75 mole % water, or at least about 90 mole % water, or about 100 mole % water.

In some embodiments, a CO₂ capture unit employs an electro-swing mechanism for capturing and later releasing CO₂. In certain cases, an electro-swing carbon dioxide unit comprises a faradaic adsorption system comprising an electrochemical cell that exploits the reductive addition of CO₂ to a redox species such as a quinone (e.g., 2,6-di-tert-butyl-1,4-benzoquinone), 4,4′-bipyridine, or a thiolate, for carbon dioxide capture. These redox agents may be provided in an organic electrolyte. In some cases, an electro-swing adsorption system provides carbon dioxide capture materials on a solid support such as a carbon nanotube support and/or a zeolite support. In some cases, an electro-swing CO₂ capture unit releases CO₂ by providing heat (e.g., by Joule heating) to an absorbent and/or electrode holding the captured CO₂.

Depending on the configuration of the CO₂ capture subsystem and its operating conditions, it can produce CO₂ from air at a high concentration of, e.g., about 90 mole % or greater. In some cases, the CO₂ capture subsystem is configured to produce CO₂ at a relatively lower concentration, which is still sufficient for CO₂ reduction electrolyzers to operate.

As examples, CO₂ capture sorbents and associated subsystem components are available from Climeworks AG of Zurich, Switzerland, Global Thermostat of New York, NY, Carbon Engineering Ltd. of Squamish, B.C., Canada, and Silicon Kingdom Holdings of Dublin, Ireland.

As indicated, captured and subsequently released CO₂ is feedstock that is delivered directly or indirectly to the cathode side of the CO₂ reduction electrolyzer. In certain embodiments, water captured from the air is also used in the feedstock of the CO₂ electrolyzer.

In certain embodiments, an air capture CO₂ electrolysis system is configured to operate in a manner that delivers CO₂ from direct air capture subsystem in a substantially pure stream of, e.g., about 99 mole % CO₂ or greater. In certain embodiments, the system is configured to operate using a lower concentration of CO₂ to the electrolyzer, e.g., about 98 mole % CO₂ or greater, or about 90 mole % CO₂ or greater, or even about 50 mole % CO₂ or greater. In some cases, quite low CO₂ concentrations are used as the feedstock. Such concentrations are still substantially greater than the atmospheric concentration of carbon dioxide, which is about 0.035 mole %. In certain embodiments, the system is configured to operate using a CO₂ concentration of about 5-15 mole %, which is mixed with air or another gas such as nitrogen.

Depending on the type of sorbent used in the process, water may also be captured along with CO₂ and released with it. In certain embodiments, the output of the CO₂ capture subsystem is humidified CO₂ having a water concentration about 0 to 20 mole % water.

In certain embodiments, the output of the CO₂ capture subsystem contains only CO₂ and other components in air such as nitrogen, oxygen, water, argon, or any combination. In all cases, the CO₂ is present at a concentration that is greater than its concentration in air. In certain embodiments, the output of the CO₂ capture subsystem contains no sulfur.

A direct air capture unit and CO₂ electrolyzer can be integrated in several ways depending on the type of air capture technology. Heat and mass transfer components may be integrated in the overall air capture CO₂ electrolysis system.

For example, in some designs, CO₂ reduction electrolyzer is configured to receive CO₂ from and provide heat and/or humidity to the direct air capture subsystem. The provided heat may release captured CO₂ during phase 2 of a direct air capture subsystem employing a temperature swing desorption mechanism. Humidified electrolyzer product gas can be used to release captured CO₂ during phase 2 of a direct air capture subsystem employing a moisture swing desorption mechanism.

In certain embodiments, the CO₂ electrolyzer is designed or configured to receive dilute CO₂ (e.g., no greater than about 50 mole % CO₂) as an input.

Direct air capture units can be designed with multiple sorbent vessels. To receive a continuous stream of CO₂ (and optionally water) from the air capture subsystem, at least two different vessels are operated to be at a different stage of sorption/desorption during operation of the overall air capture CO₂ electrolysis system. For instance, while one sorbent vessel is taking in air to capture CO₂, another may be heated to release CO₂; as each vessel continues through the sorption/desorption cycle, the sorption vessel that was taking in CO₂ will vent CO₂ and vice versa. The addition of many vessels at different points in the cycle can deliver a continuous stream of inputs to the CO₂ electrolyzer and accept a continuous stream of air containing CO₂ and moisture and/or heat and/or vacuum.

Direct air capture units can be sized to deliver the desired volume of CO₂ flow for a downstream process, such as a CO₂ electrolyzer. This may involve employing multiple sorbent-containing vessels. For example, a direct air capture subsystem may be configured to deliver 750 slpm CO₂. Such subsystem may couple to a 200-cell electrochemical stack composed of 1000 cm2 membrane-electrode assemblies operated at 300 mA/cm2 and 3 V/cell to produce 378 slpm CO and 42 slpm hydrogen given 90% CO₂ to CO current efficiency of the process. Unreacted CO₂ at the outlet of the electrolyzer may be recycled to the inlet to increase carbon efficiency. Operated continuously, the combined air capture and electrolyzer unit may produce approximately 675 kg/day CO. In general, in some designs, an air capture CO₂ electrolyzer system is configured to output at least about 100 kg/day CO and/or other CO₂ reduction product(s). in some designs, an air capture CO₂ electrolyzer system is configured to output at least about 500 kg/day CO and/or other CO₂ reduction product(s).

In certain embodiments, systems employing a carbon oxide electrolyzer and optionally a direct air capture of carbon dioxide unit also include a module configured to capture water from air or an atmosphere. In some embodiments, the module configured to capture water form air utilize solar energy from photovoltaics and/or thermal solar along with hygroscopic material. In certain embodiments, the module configured to capture water is an ambient dehumidifier such as a hydropanel (available from, e.g., Zero Mass Water, Inc. of Scottsdale, AZ).

### 3. Metal Formate Production

Returning to FIG. 1, as illustrated in operation 100, a product gas from a carbon dioxide reactor 120 of the disclosure such as carbon monoxide can be used in one or more downstream processes, including metal formate synthesis 140. In some embodiments, intermediates, reactants and/or byproducts can be re-used by being isolated, purified, and recycled back into the system. Examples include nitrogen 146, methanol 142 and/or methyl formate 144. In some embodiments, nitrogen and methanol are recycled.

In some implementations, the formate is an alkali metal formate such as sodium, potassium, or cesium formate, or an alkali earth formate such as calcium or barium formate. In certain embodiments, the formate is sodium formate. In certain embodiments, the formate is potassium formate.

Optionally, the metal formate produced can further be used as a reactant in an oxalic acid synthesis process 160 to form oxalic acid. In certain embodiments, a carbon dioxide electrolyzer and associated oxalic acid production units are deployed at or near a plant for producing cement. Carbon dioxide produced by the cement plant may be used as feedstock for the carbon dioxide electrolyzer. The oxalic acid produced by the system may be used for curing cement. In certain embodiments, oxalic acid used in cement produces calcium oxalate, which has a very low solubility. Cements produced with oxalic acid may resist degradation due to contact with acids while in use (e.g., after installation or construction).

Operation 100 may produce oxalic acid in an impure form. Therefore, the system may be further configured to provide the oxalic acid product to a separator, which may be configured to purify the oxalic acid and return unreacted alcohol to the reactor. In certain embodiments, the separator is configured to perform an azeotropic distillation on the oxalic acid product from the reactor.

Oxalic acid may be produced from a metal formate by conversion to a metal oxalate and subsequent acidification. In some implementations, a process may include the following operations: (1) produce a metal formate from carbon monoxide produced by a carbon dioxide electrolyzer (2) produce a metal oxalate from the metal formate by, e.g., pyrolysis, and (3) produce oxalic acid by exposing the metal oxalate to acid. The overall process can be conducted as a batch process. In some cases, at least the metal formate production operation and the metal oxalate production operation are performed in the same vessel. In some examples, a formate and/or oxalate production vessel may be a pressure vessel such as an autoclave. In some cases, a metal formate production vessel and/or a metal oxlate production vessel includes mechanism for reducing the particle size of a solid reactant such as a metal hydroxide. In some examples, the production vessel includes a ball mill.

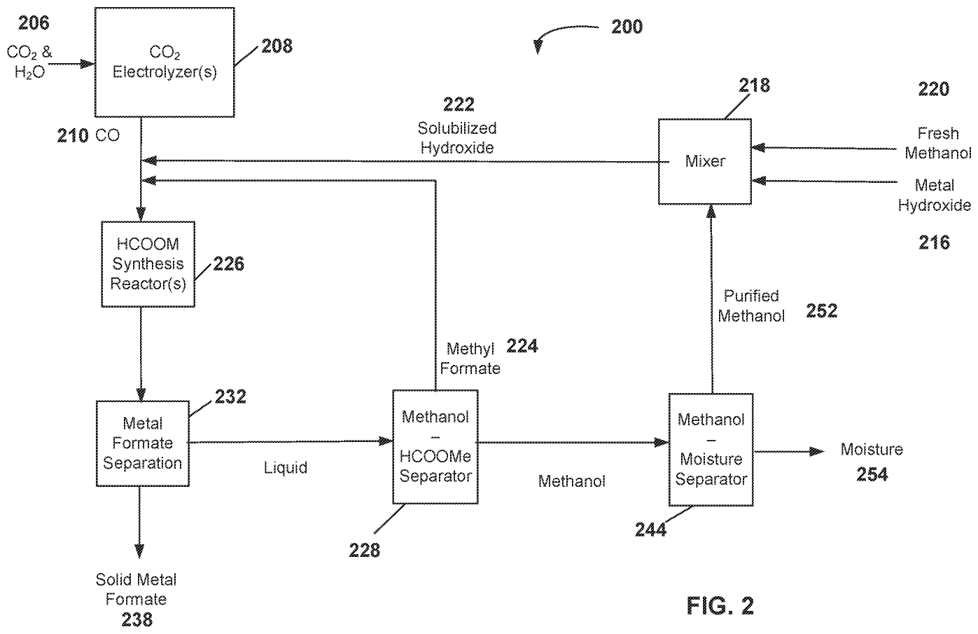

FIG. 2 schematically illustrates certain embodiments of the invention. System 200 produces a metal formate product 238 from carbon monoxide 210 generated by a carbon dioxide electrolyzer 208. Starting materials 206 for the carbon dioxide electrolyzer 208 are carbon dioxide and water.

In metal formate synthesis, the reactants are the carbon monoxide output of the carbon dioxide electrolyzer 208 and a metal hydroxide. Methanol 220 and metal hydroxide 216 are fed into mixer 218 to form solubilized hydroxide 222. The alkali metal hydroxide used in the process of the invention can be used as a solid or as a solution in a suitable solvent. It is possible to use mixtures of two or more alkali metal hydroxides. For the purpose of the present invention, the term alkali metal hydroxide encompasses both one alkali metal hydroxide and also mixtures of two or more alkali metal hydroxides. However, preference is given to using only one alkali metal hydroxide.

The alkali metal hydroxide can be lithium hydroxide, sodium hydroxide, potassium hydroxide, rubidium hydroxide or cesium hydroxide.

In some metal formate syntheses, the carbon monoxide is provided to a reactor 226 in a concentration of at least about 0.5 mole fraction, or at least about 0.8 mole fraction, or at least about 0.9 mole fraction.

Carbon monoxide 210 and solubilized hydroxide 222 are both fed into one or more metal formate synthesis reactors 226. A suitable metal formate separation process 232 is then utilized to recover solid metal formate product 238, and a liquid which includes methanol and methyl formate 224. The liquid is fed into a separator 228. Upon separation, methyl formate 224 may be fed back to the formate synthesis reactors, while recovered methanol may be purified by various techniques such as moisture separation 244 to produce moisture 254 and purified methanol 252. The purified methanol is then recycled back into the production process by introduction into mixer 218. When two or more metal formate synthesis reactors are employed, they may be arranged in a parallel arrangement, serial arrangement, or a combination of parallel and serial arrangement.

FIG. 2 illustrates the system including methanol as a solvent. Methanol is a suitable solvent for sodium formate production. However, when potassium formate or calcium formate is the desired output, water may be utilized as the solvent instead of methanol in some embodiments. Other than substituting the solvent, the system setup and product recovery is the same as that described with respect to FIG. 2.

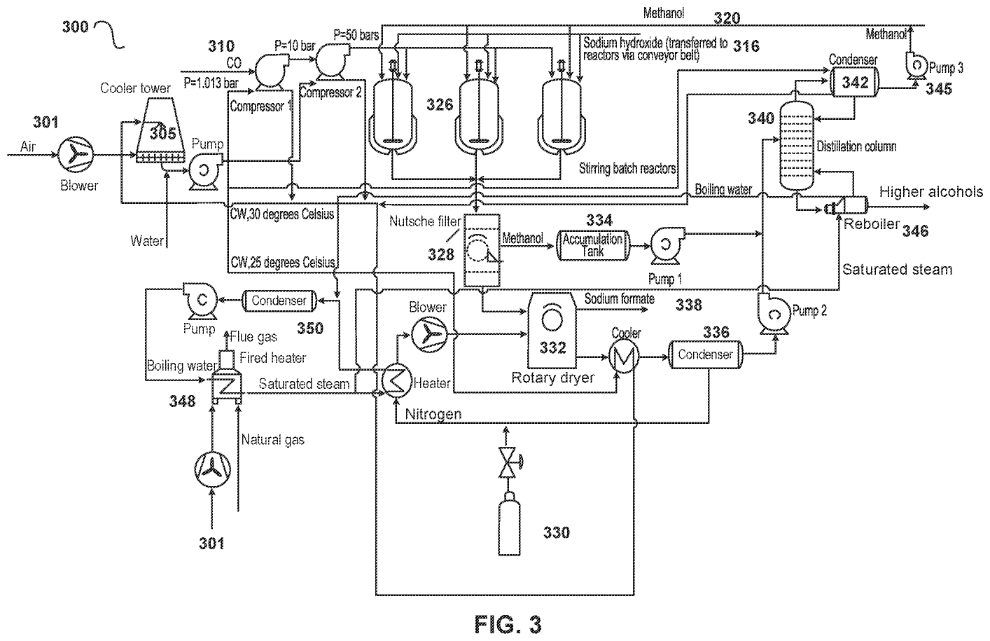

FIG. 3 illustrates an embodiment of the formate production system 300 comprising formate production reactors 326, various downstream formate recovery units and intermediate/byproduct recycling units. System 300 is configured to transport carbon monoxide gas output 310 from an electrolyzer to at least one formate production reactor 326, with stepwise pressurization by compressors from about 1, to about 10, to about 50 bars.

Air 301 is introduced into the system and passed through cooling tower 305 and then circulated through compressors which perform stepwise pressurization of carbon monoxide 310.

The reactor 326 is configured to receive not only the carbon monoxide from an electrolyzer but also metal hydroxide, solvent, and catalyst. In some embodiments, the metal hydroxide is sodium hydroxide. Solid hydroxide may be provided in various forms such as a powder. In some cases, to increase surface area of the hydroxide available for reaction, it is milled, pulverized, or otherwise reduced in particle size, optionally during reaction with carbon monoxide. For example, solid hydroxide may be ground in a ball mill autoclave during contact with carbon monoxide.

In an embodiment, system 300 is configured to feed methanol 320 solvent into the production reactors 326, and to feed solid sodium hydroxide 316 into the production reactors 326 via a conveyor belt. In some embodiments, solubilization of sodium hydroxide in methanol occurs in reactors 326.

The formate production reactor 326 may be one or more reactors which are equipped with agitators in some embodiments. The reactors may be stirring batch reactors and may be utilized all at the same time or sequentially. The agitator in the reactor may be properly sized and turned on during the reaction time to enhance the surface of the vapor and liquid phase contact.

The sodium hydroxide starting material 316 is solubilized with methanol 320 and reacts with the carbon monoxide in the formate production reactors.

In certain embodiments, a carbon dioxide electrolyzer located upstream from a metal formate production reactor is configured to operate in (a) a high reduction product to hydrogen product stream operating parameter regime as described herein, and/or (b) a high reduction product to CO₂ ratio operating parameter regime as described herein.

System 300 is configured to transport a formate-containing solution collected from the reactor or reactors 326 to a separator unit which may include filter 328 and/or dryer 332. The filter and dryer may be individual pieces of equipment or combined into one piece of equipment. The output of the filter includes wet sodium formate which may be dried for example in a rotary dryer 332.

A carrier gas such as nitrogen 330 is passed through dryer 332 to assist in the drying process. Dry sodium formate 338 is collected from the dryer 332. In some embodiments, system 300 includes a recycle path to recycle nitrogen including condenser 336.

In some embodiments, system 300 includes a recycle path to recover and reuse methanol. At filter 328, methanol may be recovered, collected in accumulation tank 334, and then pumped into distillation column 340. Methanol output from the distillation column is condensed in condenser 342 and then pumped via pump 345 into the delivery lines carrying methanol 320 into reactor 326. Additionally, in some embodiments, higher alcohol byproducts 346 are present. They may be recovered by separating them out in distillation column 340. Air 301 is also introduced into the system along with water and heated at heater 348 to provide a source of heat. Water vapor from the system can be condensed by condenser 350.

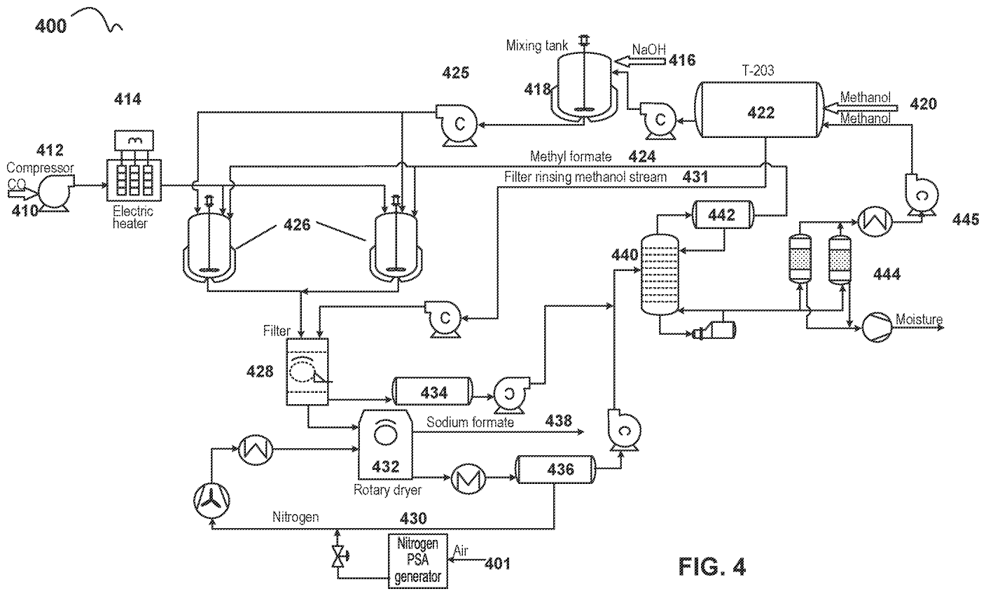

FIG. 4 is an illustration of an embodiment of the invention featuring three recycle paths: at least one for recycling carrier gas, at least one for recycling methanol and at least one for recycling methyl formate when it is present. System 400 is configured to transport carbon monoxide gas output 410 from an electrolyzer through compressor 412 and heater 414 to at least one formate production reactor 426.

In some embodiments, the carbon monoxide reactant 410 is heated to temperatures higher than about 100° C. prior to introduction into the reactor 426. Such pre-heating may be beneficial to enable favorable temperatures at the very beginning of the reaction. In some embodiments, the gas 410 may be introduced to the reactor using a specialized nozzle (e.g. a spiral spray nozzle or a cone nozzle) or other distributive means that further increases the surface area of carbon monoxide bubbles and improves the absorption rate.

In certain embodiments, the pressure of carbon monoxide may be about 5 MPa. In some embodiments, a higher carbon monoxide concentration increases the reaction rate and decreases the batch time. Higher pressures may be undesirable, as they may cause higher compressor operating costs and higher reactor capital costs.

The metal hydroxide reactant, such as sodium hydroxide 416 is introduced into a mixing tank 418 where it is solubilized in methanol 420. The methanol 420 passes through accumulation tank 422 and is then pumped into the mixing tank 418. Compressor 425 is utilized to assist with pumping recycled methanol into formate production reactors 426. In some embodiments, methanol is not pre-heated prior to introduction into reactors 426 so that it can remain in the liquid phase.

Two or more formate production reactors 426 may be utilized in process 400. In some embodiments where there are at least two reactors, they may be operated in a parallel batch mode. So doing may be advantageous because considerable residue time is needed to achieve complete conversion of sodium hydroxide molecules. The two or more reactors may operate alternatively so that downstream processes can operate in a quasi-continuous manner.

The reaction between the reactants carbon monoxide 410 and sodium hydroxide 416 in reactors 426 may take place in a temperature range of 60° C. to 200° C. in some embodiments. The reaction temperature range in some embodiments is about 100° C. to about 200° C. Since the reaction is highly exothermic (−98.3 kJ/mol), the amount of methanol in the reactor should be as small as possible to absorb enough heat and overreach 100° C. In some embodiments, the pressure may be from about 50 bar to about 100 bar. In some embodiments, the sodium hydroxide concentration is about 10 to about 25 weight percent in methanol. In some embodiments, the sodium hydroxide concentration is about 13 to about 23 weight percent in methanol, in order to avoid the need for heating or cooling during the reaction.

In some embodiments, a formate production reaction (e.g., a reaction between carbon monoxide and solid sodium hydroxide) has a residence time of at least about 15 to 60 minutes or about 20 to 40 minutes.

A limiting factor of the above-mentioned reaction is carbon monoxide's absorption into the sodium hydroxide containing methanol solution. Methods which increase the carbon monoxide absorption rate may improve the yield of the reaction, since carbon monoxide's diffusion rate into methanol is insignificant.

System 400 is configured to transport formate-containing solution collected from the reactor or reactors 426 to a separator unit which may include filter 428 and/or dryer 432. The filter and dryer may be individual pieces of equipment or combined into one piece of equipment. The output of the filter includes wet sodium formate which may be dried for example in a rotary dryer 432.

A carrier gas such as nitrogen 430 is passed through dryer 432 to assist in the drying process. Dry sodium formate 438 is collected from the dryer 432. In some embodiments, system 400 includes a recycle path to recycle nitrogen including condenser 436. Air 401 is introduced into a pressure swing absorber (PSA) generator, and nitrogen is the PSA generator output.

In some embodiments, system 400 includes a recycle path to recover and reuse methanol. At filter 428, methanol may be recovered, collected in accumulation tank 434, and then pumped into distillation column 440. Methanol output from the distillation column is passed through a moisture removal unit 444. In some embodiments, the moisture removal unit is one or more beds packed with desiccant particles. The packed bed may be filled with highly selective adsorbents including synthetic zeolites with small pores such as zeolite NaA; or with molecular sieves; or with organometallic frameworks. After passing through the moisture removal unit 444, the methanol is purified and ready for recirculation. The recovered methanol then passes through pump 445 and then fed into accumulation tank 422. Filter rinsing methanol stream 431 is a recycle conduit for recycling methanol to filter 428.

In some embodiments, system 400 includes a recycle path to recover and reuse methyl formate byproduct. The methyl formate is an intermediary product of the primary reaction in the process. Commonly between 5 and 10% of the filter outlet stream is composed of methyl formate. The methyl formate may be recovered by distillation 440 and recirculated directly to the reactor. In some embodiments, a temperature of at least about 40° C. at the highest column tray ensures that methyl formate ends up in the distillate. The distillate passes through condenser 442 and is then delivered to reactors 426.

Methyl formate 424 is not sent to mixing tank 418 because it could react with sodium hydroxide and form sodium formate. The sodium formate may then undesirably precipitate out in the mixing tank.

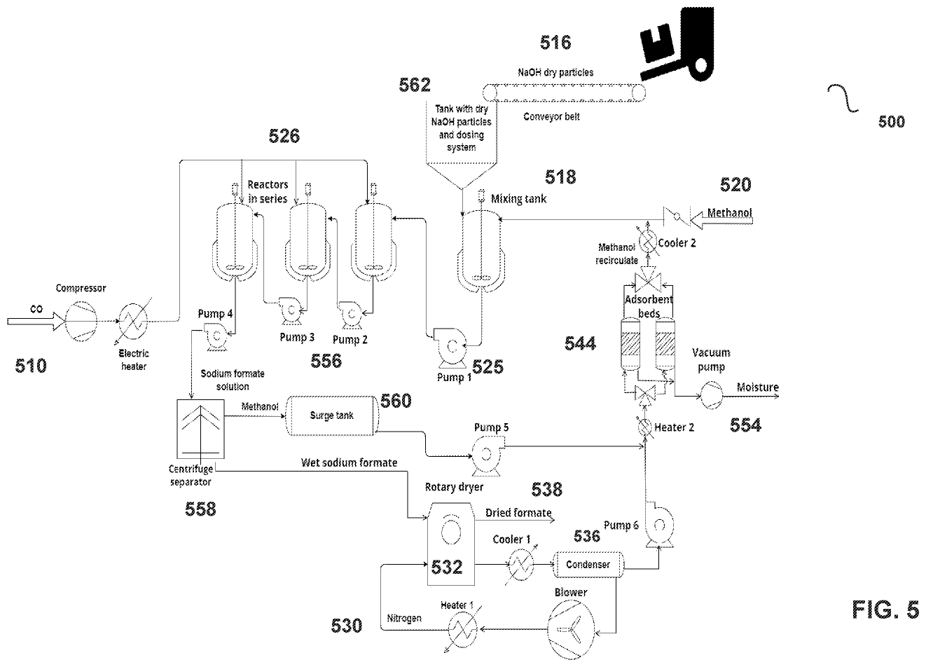

FIG. 5 is a schematic diagram for an embodiment of the invention which is a continuous process 500 including recycle paths for methanol and dryer carrier gas.

Reactant carbon monoxide gas 510 produced by a carbon dioxide electrolyzer passes through a compressor and a heater and is then fed into formate synthesis reactors 526. Reactant sodium hydroxide dry particles 516 are delivered by conveyor belt to holding tank 562 equipped with a dosing system. The appropriate amount of sodium hydroxide may then be metered into mixing tank 518 equipped with an agitator. Methanol 520 for solubilizing the sodium hydroxide is also introduced into mixing tank 518.

After mixing, the solubilized sodium hydroxide in methanol is pumped via pump 525 into the reactors 526. The reactors 526 may be connected in series via pumps 556.

The product solution exits the reactors 526 and is transported to centrifuge separator 558. Upon centrifugation, methanol and wet sodium formate are separated from each other. The methanol passes through surge tank 560 and pumped into a moisture removal unit 544 such as absorbent beds. Moisture 554 is removed, and the purified and recovered methanol is recirculated back into mixing tank 518 after cooling.

The wet sodium formate exiting centrifuge separator 558 is dried in a dryer 532 such as a rotary dryer with the assistance of a carrier gas such as nitrogen 530. Dried sodium formate product 538 is then collected, while the carrier gas exiting the dryer is passed through a condenser 536 and prepared for re-use in the dryer.

As described above, calcium formate may be synthesized using the same system discussed in the preceding paragraphs. Continuous calcium formate production is a three-step process in certain embodiments, including 1) conversion of carbon monoxide (sourced from a carbon dioxide reduction electrolyzer) to methyl formate in the presence of methanol; 2) hydrolysis of methyl formate to formic acid in the presence of water; and 3) reaction of formic acid with calcium carbonate to form calcium formate.

The first step of the process occurs in a tubular packed bed reactor at an elevated temperature and pressure. In some embodiments, the pressure is from about 5 to about 15 bar, and the temperature is from about 70° C. to about 150° C. Distillation separates the methyl formate from methanol in this step.

The second step of the process is a hydrolysis, wherein a methanol byproduct is formed which can be circulated back into the reactor as a reagent of the first step. Distillation separates the formic acid from water in this step, to prepare formic acid in a concentration of from about 15 to about 70 weight percent.

The third step of the process results in calcium formate, under reaction conditions of atmospheric temperature and pressure with excess formic acid in order to increase the reaction rate and drive the reaction to completion. The liquid phase recovered from a centrifuge containing dissolved calcium formate and excess formic acid may be recirculated back to the reactor; while CO₂ emitted can be recirculated back to the electrolyzer.

Potassium formate may be produced in the same manner as described for calcium formate above; except that potassium carbonate is substituted for calcium carbonate in the third step.

### 4. Controller Embodiments

A metal formate production system such as depicted in the figures herein may employ a control system that includes one or more controllers and one or more controllable components such as pumps, sensors, dispensers, valves, and power supplies. Examples of sensors include pressure sensors, temperature sensors, flow sensors, conductivity sensors, voltmeters, ammeters, electrolyte composition sensors including electrochemical instrumentation, chromatography systems, optical sensors such as absorbance measuring tools, and the like. Such sensors may be coupled to inlets and/or outlets of an MEA cell (e.g., in a flow field), in a reservoir for holding anode water, pure water, salt solution, etc., and/or other components of an electrolytic carbon oxide reduction system.

Among the various functions that may be controlled by one or more controllers are: flow of materials into and/or out of a module such as a reactor, a drier, a filter, a distillation column, a pump, and the like. Similarly, one or more controllers may control the temperature, pressure, and/or other process parameter of such module. In the context of a carbon dioxide electrolyzer, functions controlled by one or more controllers include: applying current and/or voltage to a carbon oxide reduction cell, controlling backpressure on an outlet from a cathode on such cell, supplying purge gas to a cathode inlet, delivering carbon oxide to the cathode inlet, humidifying carbon oxide in a cathode feed stream, flowing anode water to and/or from the anode, and controller anode feed composition. Any one or more of these functions may have a dedicated controller for controlling its function alone. Any two or more of these functions may share a controller. In some embodiments, a hierarchy of controllers is employed, with at least one master controller providing instructions to two or more component controllers. For example, a system may comprise a master controller configured to provide high level control instructions to (i) a power supply to a carbon oxide reduction cell, (ii) a cathode feed stream flow controller, and (iii) an anode feed stream flow controller.

A controller may include any number of processors and/or memory devices. The controller may contain control logic such software or firmware and/or may execute instructions provided from another source. The instructions may be executed by computer-executable components such as those integrated with a communication system. The computer-readable medium may be any suitable computer readable media such as RAMs, ROMs, flash memory, EEPROMs, optical devices (CD or DVD), hard drives, floppy drives, or any suitable device.

A controller may be integrated with electronics for controlling operation of any component, including an electrolytic cell, before, during, and after a unit operation such as reducing a carbon oxide. The controller may control various components or subparts of one or multiple electrolytic carbon oxide reduction systems. The controller, depending on the processing requirements and/or the type of system, may be programmed to control any of the processes disclosed herein, such as delivery of gases, temperature settings (e.g., heating and/or cooling), pressure settings, power settings (e.g., electrical voltage and/or current delivered to electrodes of an MEA cell), liquid flow rate settings, fluid delivery settings, and dosing of purified water and/or salt solution. These controlled processes may be connected to or interfaced with one or more systems that work in concert with the electrolytic carbon oxide reduction system.

In various embodiments, a controller comprises electronics having various integrated circuits, logic, memory, and/or software that receive instructions, issue instructions, control operations described herein. The integrated circuits may include chips in the form of firmware that store program instructions, digital signal processors (DSPs), chips defined as application specific integrated circuits (ASICs), and/or one or more microprocessors, or microcontrollers that execute program instructions (e.g., software). Program instructions may be instructions communicated to the controller in the form of various individual settings (or program files), defining operational parameters for carrying out a process on one or more components of an electrolytic carbon oxide reduction system. The operational parameters may, in some embodiments, be part of a recipe defined by process engineers to accomplish one or more processing steps during generation of a particular reduction product such as carbon monoxide, metal formate, methyl formate, and/or other compound. In one example, a programmable logic controller (PLC) controls individual components of the system.

The controller, in some implementations, may be a part of or coupled to a computer that is integrated with, coupled to the system, otherwise networked to the system, or a combination thereof. For example, the controller may utilize instructions stored remotely (e.g., in the “cloud”) and/or execute remotely. The computer may enable remote access to the system to monitor current progress of unit operations such as electrolysis operations, examine a history of past operations, examine trends or performance metrics from a plurality of operations, to change parameters of current processing, to set processing steps to follow a current processing, or to start a new process. In some examples, a remote computer (e.g., a server) can provide process recipes to a system over a network, which may include a local network or the internet. The remote computer may include a user interface that enables entry or programming of parameters and/or settings, which are then communicated to the system from the remote computer. In some examples, the controller receives instructions in the form of data, which specify parameters for each of the processing steps to be performed during one or more operations.

The controller may be distributed, such as by comprising one or more discrete controllers that are networked together and working towards a common purpose, such as applying current to an MEA cell and other process controls described herein. An example of a distributed control system for such purposes includes one or more processors on a system for electrolytically reducing a carbon oxide and one or more processors located remotely (such as at the platform level or as part of a remote computer) that combine to control a process.

Controllers and any of various associated computational elements including processors, memory, instructions, routines, models, or other components are sometimes described or claimed as “configured to” perform a task or tasks. In such contexts, the phrase “configured to” is used to denote structure by indicating that the component includes structure (e.g., stored instructions, circuitry, etc.) that performs a task or tasks during operation. As such, a controller and/or associated component can be said to be configured to perform the task even when the specified component is not necessarily currently operational (e.g., is not on).

Controllers and other components that are “configured to” perform an operation may be implemented as hardware—for example, circuits, memory storing program instructions executable to implement the operation, etc. Additionally, controllers and other components “configured to” perform an operation may be implemented as hardware that is manipulated by software and/or firmware (e.g., an FPGA or a general-purpose processor executing software) to operate in manner that is capable of performing the recited task(s). Additionally, “configured to” can refer to one or more memories or memory elements storing computer executable instructions for performing the recited task(s). Such memory elements may include memory on a computer chip having processing logic.

Non-computation elements such as reactors such electrolyzers, membrane assemblies, layers, and catalyst particles may also be “configured” to perform certain functions. In such contexts, the phrase “configured to” indicate that the referenced structure has one or more features that allow the function to be performed. Examples of such features include physical and/or chemical properties such as dimensions, composition, porosity, etc.

### CONCLUSION

Although omitted for conciseness, embodiments of the system and/or method can include every combination and permutation of the various system components and the various method processes, wherein one or more instances of the method and/or processes described herein can be performed asynchronously (e.g., sequentially), concurrently (e.g., in parallel), or in any other suitable order by and/or using one or more instances of the systems, elements, and/or entities described herein.

The Figures illustrate the architecture, functionality and operation of possible implementations of systems, methods and computer program products according to disclosed embodiments, example configurations, and variations thereof. In this regard, each block in a block diagram may represent a module, a group of modules, a segment or a module, and may embody one or more steps or operations. It should also be noted that, in some alternative implementations, the functions noted in the block can occur out of the order noted in the figures. For example, two blocks shown in succession may, in fact, be executed substantially concurrently, or the blocks may sometimes be executed in the reverse order, depending upon the functionality involved.

Although the foregoing embodiments have been described in some detail for purposes of clarity of understanding, it will be apparent that certain changes and modifications may be practiced within the scope of the appended claims. It should be noted that there are many alternative ways of implementing the processes, systems and apparatus of the present embodiments. Accordingly, the present embodiments are to be considered as illustrative and not restrictive, and the embodiments are not to be limited to the details given herein.

As a person skilled in the art will recognize from the previous detailed description and from the figures and claims, modifications and changes can be made to the disclosed embodiments of the disclosure without departing from the scope of this disclosure defined in the following claims.

What is claimed is:

1. A method of producing a metal formate comprising:
(a) reducing carbon dioxide to carbon monoxide in a carbon dioxide reduction electrolyzer comprising a membrane electrode assembly, which comprises one or more ion conductive polymer layers and a cathode catalyst for facilitating chemical reduction of carbon dioxide to carbon monoxide;
(b) reacting carbon monoxide produced by the carbon dioxide reduction electrolyzer with a metal hydroxide in methanol in at least one formate synthesis reactor to produce a metal formate;
(c) separating and purifying the metal formate produced in (b); and
(d) recycling methanol to the at least one formate synthesis reactor.

2. The method of claim 1, wherein the at least one formate synthesis reactor comprises at least two batch formate synthesis reactors and wherein reacting carbon monoxide produced by the carbon dioxide reduction electrolyzer with a metal hydroxide in methanol is performed alternately in the at least two batch formate synthesis reactors.

3. The method of claim 1, wherein the at least one formate synthesis reactor comprises at least two batch formate synthesis reactors and wherein reacting carbon monoxide produced by the carbon dioxide reduction electrolyzer with a metal hydroxide in methanol is performed continuously in the at least two batch formate synthesis reactors.

4. The method of claim 1, wherein reacting the carbon monoxide with a metal hydroxide takes place at a reaction temperature of about 60° C. to about 200° C., and at a pressure of about 1 MPa to about 10 MPa.

5. The method of claim 1, wherein the metal hydroxide has a concentration of about 1 to about 25 percent by weight of metal hydroxide in the methanol.

6. The method of claim 1, further comprising pre-heating the carbon monoxide from (a) before reacting in (b).

7. The method of claim 4, wherein the carbon monoxide is pre-heated to at least about 100° C.

8. The method of claim 1, wherein a reaction temperature is about 100° C. to about 200° C.

9. The method of claim 1, further comprising producing methyl formate when reacting carbon monoxide with metal hydroxide in (b).

10. The method of claim 9, further comprising recovering the methyl formate by distillation and recycling the methyl formate to the at least one formate synthesis reactor.

11. The method of claim 1, wherein (c) comprises drying metal formate with a carrier gas, and producing dry metal formate and a dryer effluent gas stream.

12. The method of claim 11, further comprising condensing liquid from the dryer effluent gas stream to form a condensed liquid.

13. The method of claim 12, further comprising distilling the condensed liquid to obtain recovered methanol.

14. The method of claim 13, wherein recycling methanol to the at least one formate synthesis reactor comprises recycling the recovered methanol to the at least one formate synthesis reactor.

15. The method of claim 1, wherein (d) further comprises moisture removal.

16. The method of claim 12, further comprising recovering an emitted carrier gas; and recycling the emitted carrier gas to a dryer.

17. The method of claim 11, wherein the carrier gas is nitrogen gas.

18. The method of claim 1, wherein the metal hydroxide is sodium hydroxide, potassium hydroxide, rubidium hydroxide, cesium hydroxide, or a combination thereof.

19. The method of claim 1, further comprising converting the metal formate to a metal oxalate.

20. The method of claim 19, further comprising contacting the metal oxalate with an acid to produce oxalic acid.
